(19) United States
(12) Patent Application Publication
(10) Pub. No.: US 2018/0014822 A1
(43) Pub. Date: Jan. 18, 2018

(54) FASCIA CLOSURE DEVICE AND METHOD

(71) Applicant: TROCARE, LLC, Houston, TX (US)

(72) Inventors: Gerald FEUER, Atlanta, GA (US); Clark B. FOSTER, Mission Viejo, CA (US)

(73) Assignee: TROCARE, LLC, Houston, TX (US)

(21) Appl. No.: 15/523,274

(22) PCT Filed: Oct. 28, 2015
(86) PCT No.: PCT/US15/57872

§ 371 Date: Apr. 28, 2017

Related U.S. Application Data
(60) Provisional application No. 62/069,686, filed on Oct. 28, 2014.

(51) Int. Cl. A61B 17/00

(52) U.S. Cl. CPC A61B 17/0057; A61B 2017/00862; A61B 2017/00623; A61B 2017/00637; A61B 2017/00659

(57) ABSTRACT

Fascial wound closure device that includes an applicator releasably attacked to an implant main body having a plurality of connecting members configured to extend from the implant main body. A plurality of tissue engagement hooks mounted within a respective one of the plurality of connecting members configured to penetratingly engage at least one layer of body tissue so as to securely close a fascial wound. Method of producing a secure closure of a laparoscopic puncture site is provided.

### CROSS-REFERENCE TO RELATED APPLICATIONS

The present application claims benefit to U.S. Provisional Application No. 62/069,686 filed Oct. 28, 2014, the entire contents of which are incorporated by reference.

### FIELD

The present disclosure relates to closing fascial wounds. In particular, the present disclosure relates to devices and methods for closing fascial wounds caused by instruments used in laparoscopic surgical procedures.

### BACKGROUND

Laparoscopic surgery generally requires the creation of one or more trocar puncture wounds through which surgical instruments, cameras, and light sources are deployed to enable the surgeon to perform the surgery. Upon removal of the laparoscopic instruments, a wound remains that passes from the exterior skin into the body, and in particular, through the fascia layer. The puncture wounds created within the body by the surgeon to gain access to the surgical site are often difficult and time-consuming to close. Further, failure to properly close a laparoscopic puncture site can lead to subsequent hernias and other medical complications.

### BRIEF DESCRIPTION OF THE DRAWINGS

In order to describe the manner in which the advantages and features of the disclosure can be obtained, reference is made to the appended drawings. The drawings presented provide only non-limiting examples of what is disclosed herein.

FIG. 1 is a cross-sectional view of a wound closure device prior to insertion or partial insertion into an abdominal cavity, according to an example of the present disclosure;

FIG. 2 is a cross-sectional view of a wound closure device following insertion or partial insertion into an abdominal cavity beyond a location of a fascial wound in a retracted configuration, according to an example of the present disclosure;

FIG. 3 is a cross-sectional view of a wound closure device with connecting members in a partially extended configuration, according to an example of the present disclosure;

FIG. 4 is a cross-sectional view of a wound closure device with connecting members in an extended configuration, according to an example of the present disclosure;

FIG. 5 is a cross-sectional view of a wound closure device with tissue engagement hooks in pressure contact with a layer of body tissue, according to an example of the present disclosure;

FIG. 6 is a cross-sectional view of a wound closure device in an engaged configuration, according to an example of the present disclosure;

FIG. 7 is a cross-sectional view of a wound closure device in an engaged configuration with the pull rod of the applicator disengaged from the implant main body, according to an example of the present disclosure;

FIG. 8 is a cross-sectional view of a wound closure device in an engaged configuration with the tethers severed and the applicator disengaged from the implant main body, according to an example of the present disclosure;

FIG. 9 is a cross-sectional view of an implant engaged with a layer of body tissue following disengagement and removal of the applicator, according to an example of the present disclosure;

FIG. 10A is a bottom perspective view of an implant partially engaged with body tissue by a plurality of tissue engagement hooks, according to an example of the present disclosure;

FIG. 10B is a bottom perspective view of an installed implant engaged with body tissue by a plurality of tissue engagement hooks, according to an example of the present disclosure;

FIG. 11 is a cross-sectional view of an implant installed to close a fascial wound by a plurality of tissue engagement hooks, according to an example of the present disclosure;

FIG. 12 is a perspective view of the distal portion of a wound closure device in the retracted configuration, according to an example of the present disclosure;

FIG. 13A is a cross-sectional view of the distal portion of a wound closure device with tissue engagement hooks in the retracted configuration, according to an example of the present disclosure;

FIG. 13B is a cross-sectional view of the distal portion of a wound closure device with tissue engagement hooks in the engaged configuration and extended away from the implant main body, according to an example of the present disclosure;

FIG. 13C is a cross-sectional view of the distal portion of a wound closure device with tissue engagement hooks in the engaged configuration and drawn toward the implant main body, according to an example of the present disclosure;

FIG. 14A is a perspective view of the distal portion of a wound closure device having a spring deployment retainer, according to an example of the present disclosure;

FIG. 14B is a perspective view of the distal portion of a wound closure device in the engaged configuration with the proximal end of a tissue engaging hook captured in the receiving space of a spring deployment retainer, according to an example of the present disclosure;

FIG. 15A is a close-up cross-sectional view of the spring deployment retainer depicted in FIGS. 14A and 14B, according to an example of the present disclosure;

FIG. 15B is a close-up cross-sectional view of the spring deployment retainer with a proximal portion of a tissue engagement hook captured within the receiving space, according to an example of the present disclosure;

FIG. 16A is a perspective view of the distal portion of a wound closure device having rotating tissue engagement hooks held in an unactuated condition by a retention device, according to an example of the present disclosure;

FIG. 16B is a perspective view of the distal portion of a wound closure device having rotating tissue engagement hooks with the retention device in the disengaged position, according to an example of the present disclosure;

FIG. 17A is a close-up cross-sectional view of a rotating tissue engagement hook in the unactuated condition, according to an example of the present disclosure;

FIG. 17B is a close-up cross-sectional view of a rotating tissue engagement hook in the engaged position, according to an example of the present disclosure;

FIG. 18A is a bottom perspective view of an implant having a plurality of rotating tissue engagement hooks in pressure contact with body tissue, according to an example of the present disclosure;

FIG. 18B is a bottom perspective view of an installed implant engaged with body tissue by a plurality of rotating tissue engagement hooks, according to an example of the present disclosure;

FIG. 19 is a cross-sectional view of an implant installed to close a fascial wound by a plurality of rotating tissue engagement hooks, according to an example of the present disclosure;

FIG. 20A is a perspective view of the distal portion of a wound closure device having pre-curled tissue engagement hooks in a retracted and biased state, according to an example of the present disclosure;

FIG. 20B is a perspective view of the distal portion of a wound closure device having pre-curled tissue engagement hooks in a deployed and unbiased state, according to an example of the present disclosure;

FIG. 21A is a cross-sectional view of the distal portion of a wound closure device having pre-curled tissue engagement hooks in a retracted and biased state, according to an example of the present disclosure;

FIG. 21B is a cross-sectional view of the distal portion of a wound closure device having pre-curled tissue engagement hooks in a deployed and unbiased state, according to an example of the present disclosure;

FIG. 22 is a perspective view of the distal portion of a wound closure device having elastic members with a tissue engagement hook formed at the end in a first configuration, according to an example of the present disclosure;

FIG. 23 is a cross-sectional view of the distal portion of a wound closure device having elastic members with a tissue engagement hook formed at the end, according to an example of the present disclosure;

FIG. 24A is a cross-sectional view of a wound closure device having elastic members, with the tissue engagement hook in a partial engagement position, according to an example of the present disclosure;

FIG. 24B is a cross-sectional view of a wound closure device having elastic members, with the tissue engagement hook in a fully engaged position, according to an example of the present disclosure; and

FIG. 25 is a perspective view of elastic members coupled to the implant main body, according to an example of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

It should be understood at the outset that although illustrative implementations of one or more examples are illustrated below, the disclosed devices and methods may be implemented using any number of techniques. The disclosure should in no way be limited to the illustrative implementations, drawings, and techniques illustrated herein, but may be modified within the scope of the appended claims along with their full scope of equivalents.

Unless otherwise specified, any use of any form of the term “couple,” or any other term describing an interaction between elements is not meant to limit the interaction to direct interaction between the elements and also may include indirect interaction between the elements described. In the following discussion and in the claims, the terms “including” and “comprising” are used in an open-ended fashion, and thus should be interpreted to mean “including, but not limited to . . . ”. “Upward” as used herein means in the direction extending from the proximal end of the applicator and/or in the direction away from the patient. Examples as used herein refer to non-limiting examples. The term “pressure contact,” as used herein, refers to any type of physical contact. For example, as described herein, pressure contact can exist between the tissue engagement hook and at least one layer of body tissue, including but not limited to, partial penetration of the tissue engagement hook into the layer of body tissue. The various characteristics described in more detail below, will be readily apparent to those skilled in the art with the aid of this disclosure upon reading the following detailed description, and by referring to the accompanying drawings.

The present disclosure provides devices and methods for producing a secure closure of a laparoscopic puncture site. More specifically, the present disclosure provides a wound closure device that includes an implant having an implant main body. Additionally, in at least one example of what is described, an applicator is provided. In other embodiments, both the applicator and implant can be implemented together. While the disclosure describes particular examples of applicators and implants having implant main bodies, the applicators can be used with other implants. Additionally, the implants having implant main bodies can be implemented with different applicators than the one described herein.

The applicator is configured to releasably engage the implant main body such that the applicator provides for positioning and activation of the implant in order to engage and close the tissue surrounding the fascial wound. The applicator is further configured to disengage from the implant main body, thereby leaving the installed implant and implant main body to maintain secure closure of the fascial wound.

The implant includes an implant main body and a plurality of connecting members each configured to extend from the implant main body. A plurality of tissue engagement hooks are mounted within each of the plurality of connecting members. During insertion or partial insertion of the wound closure device into the abdominal cavity, the distal end of the applicator is releasably attached to the implant main body and the connecting members are in a retracted configuration. After insertion of the wound closure device into the abdominal cavity beyond a location of a fascial wound, the applicator is configured to activate the implant into an engaged configuration. In the engaged configuration, the plurality of connecting members are extended from the implant main body and the plurality of tissue engagement hooks are engaged with at least one layer of body tissue. The plurality of connecting members are configured to rotate about the implant main body such that in the retracted configuration the plurality of tissue engagement hooks are closer to the proximal end of the applicator than in the engaged configuration.

The present disclosure also provides for methods of closing a fascial wound that include inserting a wound closure device into an abdominal cavity partially beyond a location of the fascial wound and extending a plurality of connecting members from the implant main body such that the connecting members rotate about the implant main body. The method further includes bringing the plurality of tissue engagement hooks into pressure contact with at least one layer of tissue and causing the plurality of tissue engagement hooks to penetrate at least one layer of body tissue. The method also includes disengaging the applicator from the implant main body.

According to at least one aspect of the present disclosure, the presently disclosed devices and methods provide for a secure closure that is less likely to result in subsequent hernia formation in a patient than many conventional devices and methods.

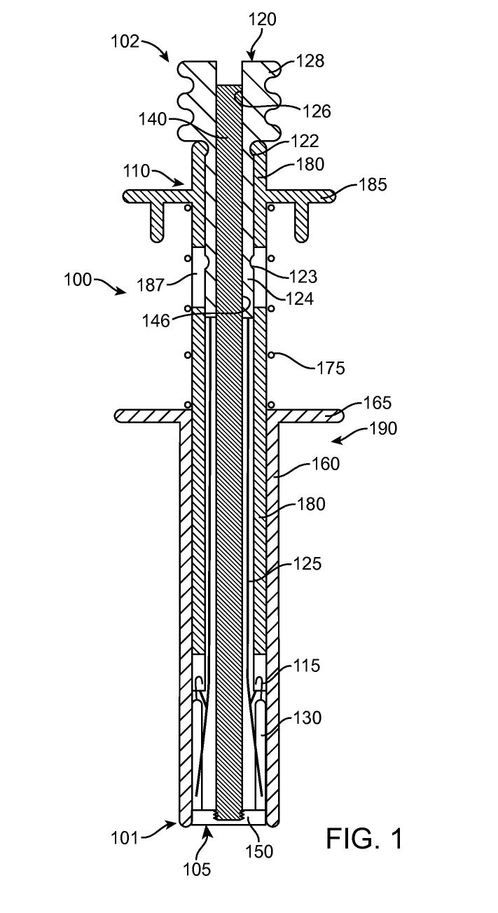

FIGS. 1-9 illustrate sequential cross-sectional views of a wound closure device 100 during insertion and installation of the implant 105 to close a fascial wound, according to an example of the present disclosure.

FIG. 1 illustrates a cross-sectional view of a wound closure device 100 prior to insertion or partial insertion into an abdominal cavity, according to an example of the present disclosure. As depicted, the applicator 110 can have a distal end 101 and a proximal end 102. Additionally, the applicator 110 is releasably attached to an implant main body 150 at the distal end 101 of the applicator 110. A plurality of connecting members 130 are coupled to the implant main body 150. Each connecting member 130 is configured to rotate about the implant main body 150 thereby extending from the implant main body 150 upon actuation by the applicator 110. Mounted within each one of the plurality of connecting members 130 is a tissue engagement hook 115. While a single tissue engagement hook 115 for each connecting member 130 is generally described and shown, the connecting member 130 can include one or more tissue engagement hooks 115. The remaining disclosure is discussed in regards to the single tissue engagement hook 115.

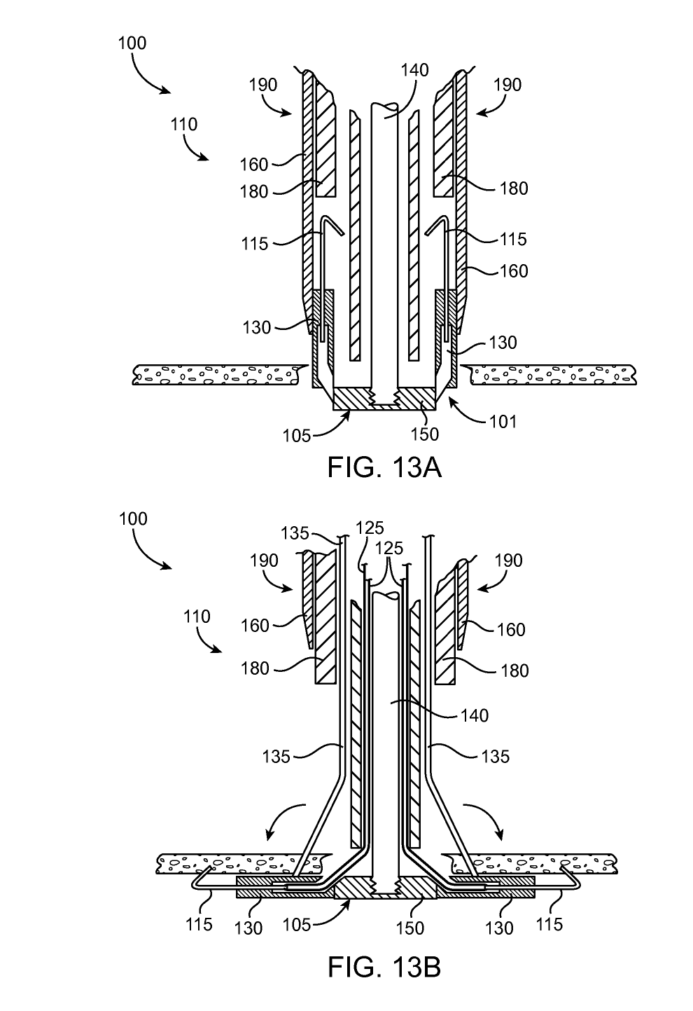

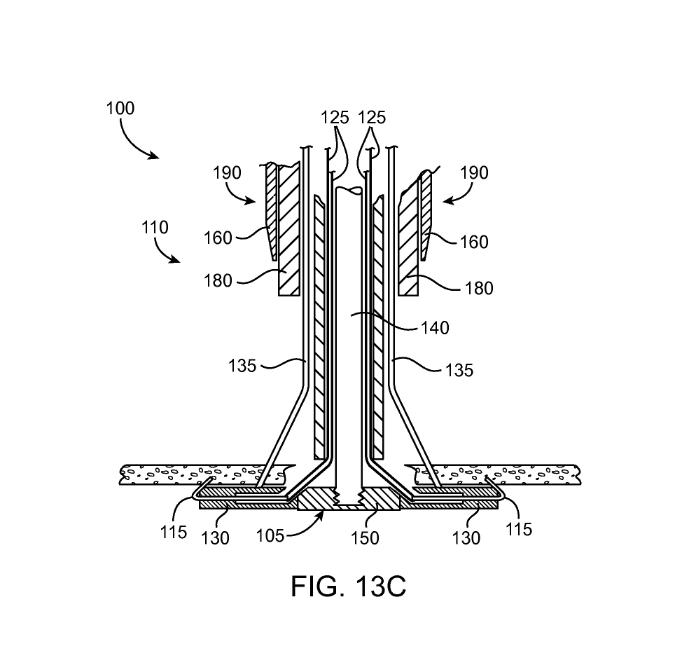

As depicted in FIG. 1, each of the plurality of connecting members 130 is coupled to the engagement mechanism 120 by a tether 125. The tether 125 provides for actuation of the plurality of tissue engagement hooks 115 by the engagement mechanism 120, thereby causing the tissue engagement hooks 115 to engage a layer of body tissue. In some cases, the tether 125 can be coupled to an actuation mechanism in the connecting member 130 configured to actuate the corresponding tissue engagement hook 115 to engage the body tissue. In some cases, the tether 125 can be coupled to an end of a tissue engagement hook 115 mounted in each of the connecting members 130, for example, as shown in FIGS. 13B and 13C. In the illustrated example, the one or more tethers 125 are coupled at a first end to the engagement mechanism 120 and coupled at a second end to the end of a tissue engagement hook 115 that is mounted within a corresponding connecting member 130. In some cases, the tether 125 may also be coupled to the applicator 110 at a second attachment site, such as the applicator main body 180, so that upon severance of the tether 125 and withdrawal of the applicator 110 from the implant main body 150, the tether 125 can remain attached to the applicator 110 for easy removal of the tether 125 from the fascial wound site. In some cases, the tether 125 may be formed into one or more loops such that severance of the tether 125 at one location along the tether 125 provides for uncoupling of the tether 125 from the tissue engagement hook 115 and easy removal of the tether 125 from the fascial wound site. In some cases, the tether 125 can be coupled to a corresponding tissue engagement hook 115 such that upon severance of the tether 125 and removal of the applicator 110 from the fascial wound site, the remaining tether 125 can serve as either a primary or secondary fascial wound closure mechanism, for example, by tying the remaining tether 125 in a similar manner as the way that sutures are used to close a wound.

In at least one aspect of the present disclosure, the engagement mechanism 120 can include a plunger 124. The engagement mechanism 120 can also in some cases include a ring collar 128. While a ring collar 128 is illustrated, the present disclosure can also be implemented with other types of structures that can be grasped, including but not limited to a flattened disc, a flange, and two or more arms. Translational movement of the engagement mechanism 120 is restricted by a first detent 122 and a second detent 123 formed between the engagement mechanism 120 and the applicator main body 180. As depicted in FIG. 1, the engagement mechanism 120 is engaged with the applicator main body 180 to form the first detent 122. The applicator main body 180 can include a main flange 185.

As depicted in FIG. 1, a securement member 190 surrounds the plurality of connecting members 130 and tissue engagement hooks 115 so as to protect the connecting members 130 and tissue engagement hooks 115. Additionally, the securement member 190 can prevent pre-mature extension of the connecting members 130 and engagement of the tissue engagement hooks 115 during insertion of the wound closure device into the abdominal cavity. As shown in FIG. 1, the securement member 190 is a retractable sleeve 160 that includes a sleeve flange 165. However, the wound closure device can include other types of securement members 190 without departing from the spirit and scope of the present disclosure. In other examples, the securement member 190 can be any structure that prevents the connecting members 130 from extending. For example, the securement member 190 can be a pin and a release member. Additionally, the securement member 190 can include a cam surface that only allows actuation based on the configuration of the cam surface. Further, in at least one embodiment, the securement member 190 may only partially surround the plurality of connecting members and/or the plurality of tissue engagement hooks without departing from the spirit and scope of the present disclosure. Additionally, a wound closure device 100 that does not include a securement member 190 is still within the spirit and scope of the present disclosure. In at least one aspect of the present disclosure, a spring 175 can couple the sleeve flange 165 with the main flange 185. The spring 175 can be configured to bias the sleeve flange 165 away from the main flange 185. In other examples, the spring 175 can be replaced by another type of biasing member that biases the sleeve flange 165 away from the main flange 185.

As depicted in FIG. 1, applicator 110 includes a pull rod 140 coupled with the engagement mechanism 120 and releasably attached to the implant main body 150. The pull rod 140 can be any elongated member including, but not limited to, a cylindrical rod, a rectangular rod, or a hexagonal rod, that can form an attachment point with the implant main body 150. While a pull rod 140 is shown as the attachment point between the applicator 110 and implant main body 150, any portion of the applicator 110 suitable to form an attachment point between the applicator 110 and the implant main body 150 may form the attachment point without departing from the spirit and scope of the present disclosure.

The applicator 110 can be releasably secured to the implant main body 150. In one example, the pull rod 140 can be releasably attached to the implant main body 150 by a screw-in mechanism, as depicted in FIG. 1. In other examples, the applicator 110 can be press fit into the implant main body 150 so that upon application of a predetermined force the applicator 110 separates from the implant main body 150. In still other examples, the applicator 110 can include a release apparatus that is configured to cause the implant main body 150 to separate from the applicator 110.

The pull rod 140 is coupled with the engagement mechanism 120 such that rotation of the engagement mechanism 120 causes rotation of the pull rod 140. As the engagement mechanism 120 is rotationally connected to the pull rod 140, rotation of the engagement mechanism 120 provides a mechanism by which the pull rod 140 can be unscrewed from the implant main body 150 thereby disengaging the applicator 110 from the implant main body 150.

As depicted in FIG. 1, the pull rod 140 is coupled with the engagement mechanism 120 by the pull rod 140 being received with an aperture of the engagement mechanism 120. For example, the engagement mechanism 120 can have an aperture surface 126 configured to matingly engage an external surface 146 of the pull rod 140 such that rotation of the engagement mechanism 120 relative to the implant main body 150 causes rotation and release of the pull rod 140 from the implant main body 150. In some cases, the aperture surface 126 of the engagement mechanism 120 and the external surface 146 of the pull rod 140 can each be hexagonal in shape. However, any mechanism for rotationally connecting the engagement mechanism 120 to the pull rod 140 may be utilized in the wound closure device 100 without departing from the spirit and scope of the present disclosure. For example, the engagement mechanism 120 and the pull rod 140 can be coupled together through a ratcheting mechanism. In one example, the ratcheting mechanism can be configured so that it requires a predetermined force to actuate

Additionally, one or more tethers 125 can be implemented so that the one or more tethers 125 are coupled to the engagement mechanism 120. As illustrated, the one or more tethers 125 can be formed as a tether loop. As illustrated, there are a plurality of tethers 125. The present disclosure can be implemented with one or more tethers 125 such that one tether 125 is configured to actuate more than one of the plurality of tissue engagement hooks 115.

As shown in FIG. 1, the applicator main body 180 can also include an aperture 187 that provides access to the one or more tethers 125. As illustrated, the one or more tethers 125 can be severed during the disengagement of the applicator 110 from the implant main body 150. For example, following installation of the implant main body 150 to close the fascial wound, the tethers 125 can be severed so as to release the tether 125 from the implant main body 150. In at least one aspect of the present disclosure, the aperture 187 provides access to the plurality of tethers 125 only upon actuation of the engagement mechanism 120. The tethers 125 can be cut using a pair of surgical scissors or other sharp object. When the tether 125 is formed as a loop, the loop can be configured such that only a single portion of the tether 125 is exposed at the aperture 187. In other examples, two portions of the tether 125 can be exposed at the aperture 187 and the surgeon or assistant would need to only sever one of the portions so that the tether could be removed.

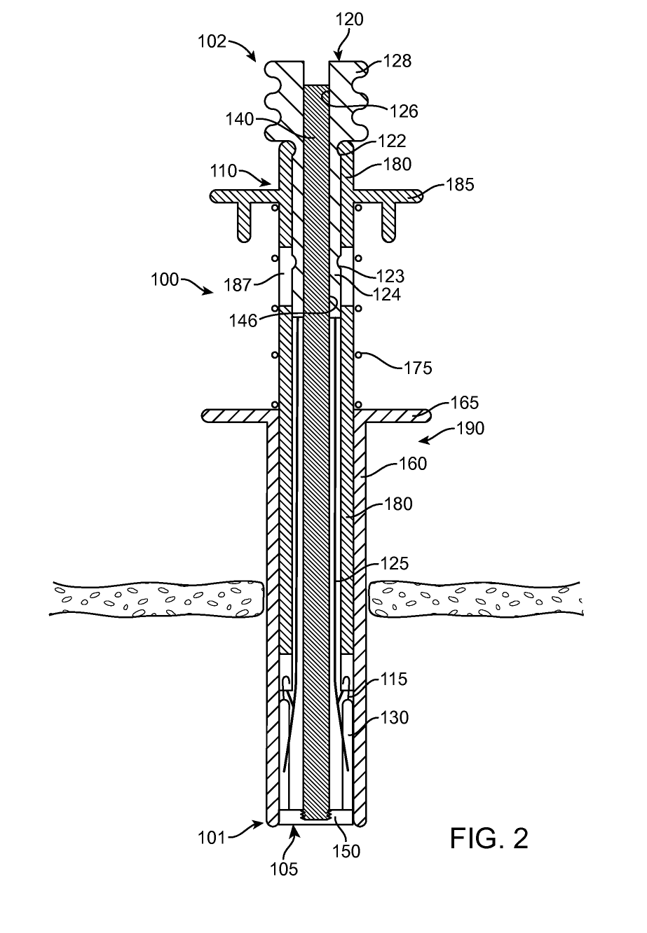

FIG. 2 illustrates a cross-sectional view of a wound closure device 100 following insertion or partial insertion into an abdominal cavity beyond a location of a fascial wound, according to an example of the present disclosure. As depicted in FIG. 2, the wound closure device 100 is in a retracted configuration during insertion of the distal end 101 of the wound closure device 100 into the abdominal cavity. In the retracted configuration, the connecting members 130 are substantially parallel to the applicator main body 180 instead of being extended from the implant main body 150. As depicted in FIG. 2, the securement member 190, in the form of a retractable sleeve 160, surrounds the plurality of tissue engagement hooks 115 in the retracted configuration.

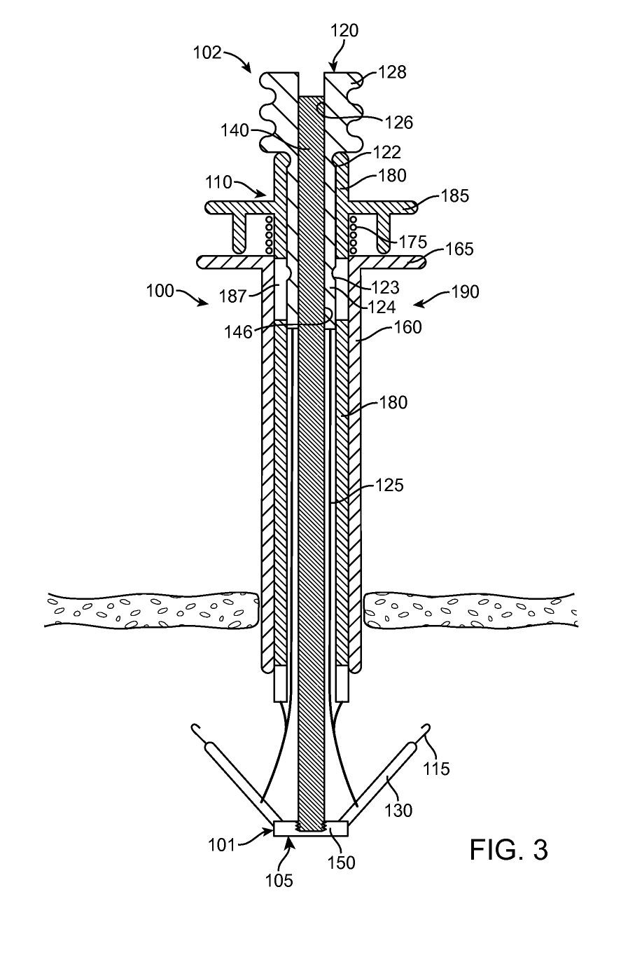

FIG. 3 illustrates a cross-sectional view of the wound closure device 100 with connecting members 130 in a partially extended configuration, according to an example of the present disclosure. As depicted in FIG. 3, the securement member 190, in the form of a retractable sleeve 160, has retracted by translating toward the proximal end 102 of the applicator 110, thereby allowing the plurality of connecting members 130 to rotate about the implant main body 150 resulting in partial extension of the connecting members 130 from the implant main body 150. In some cases, the connecting members 130 can be biased so as to rotate about the implant main body 150 and at least partially extend from the implant main body 150 upon retraction of the securement member 190. In other cases, the connecting members 130 can be coupled with a spring or biasing member that facilitates rotation of the connecting members 130 about the implant main body 150 and at least partial extension from the implant main body 150 upon retraction of the securement member 190. In one example, a spring hinge can couple the connecting members 130 to the implant main body 150. In other embodiments, a strip of elastically deformable material can be implemented to connect the connecting members 130 to the implant main body 150. The wound closure device 100 may include any type of coupling between the connecting members 130 and the implant main body 150 that allows the connecting members 130 to rotate about the implant main body 150 and at least partially extend from the implant main body 150, without departing from the spirit and scope of the present disclosure.

As depicted in FIG. 3, as the securement member 190 translates toward the proximal end 102 of the applicator 110, spring 175 is compressed. Additionally, further translational movement toward the proximal end 102 of the applicator 110 by securement member 190 is limited by eventual engagement of the sleeve flange 165 of the retractable sleeve 160 with the main flange 185 of applicator main body 180.

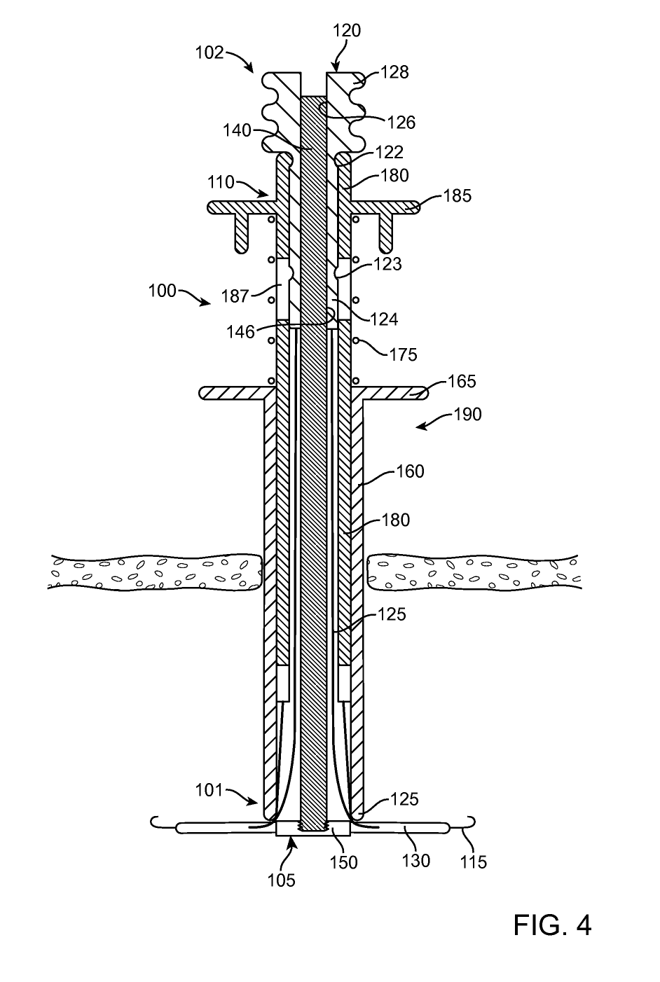

FIG. 4 illustrates a cross-sectional view of the wound closure device 100 with connecting members 130 in an extended configuration, according to an example of the present disclosure. As depicted in FIG. 4, the securement member 190, in the form of retractable sleeve 160, has translated toward the distal end 101 of the applicator 110, with the aid of spring 175, to engage the connecting members 130 so as to cause the connecting members 130 to fully extend from implant main body 150. As indicated by comparison of FIG. 4 to FIG. 2, in the retracted configuration the plurality of tissue engagement hooks are closer to the proximal end 102 of applicator 110 than in the engaged configuration.

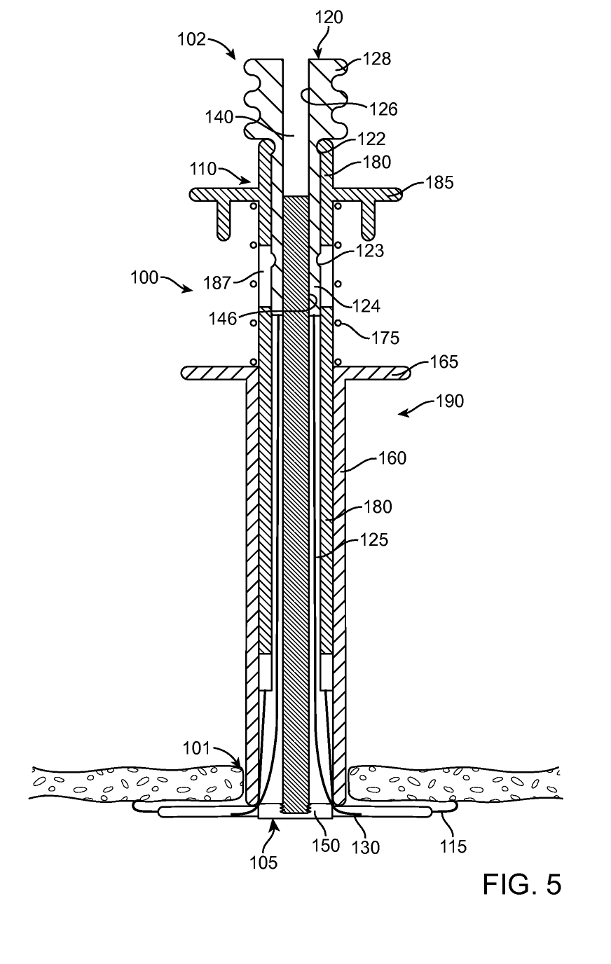

FIG. 5 illustrates a cross-sectional view of the wound closure device 100 with tissue engagement hooks 115 in pressure contact with a layer of body tissue, according to an example of the present disclosure. As depicted in FIG. 5, the wound closure device 100 has been pulled upward, by application of a pulling motion to the applicator, so as to bring the tissue engagement hooks 115 into pressure contact with at least one layer of body tissue. As depicted in FIG. 5, the engagement mechanism 120 of applicator 110 includes a ring collar with gripping surface sufficient to facilitate pulling the wound closure device 100 in the upward direction. In some cases, the engagement mechanism 120 of applicator 110 can further include a mechanical mechanism capable of bringing the tissue engagement hooks 115 in pressure contact with the body tissue (not shown) without moving the entire wound closure device 100 in the upward direction. In one example, a biased operated mechanism can be implemented so that that the proper force occurs during each installation. The biased operated mechanism can include a biasing member such as a spring or an elastically deformed material that causes the applicator to bring the tissue engagement hooks 115 into pressure contact with the body tissue upon release of a retention member such as a trigger.

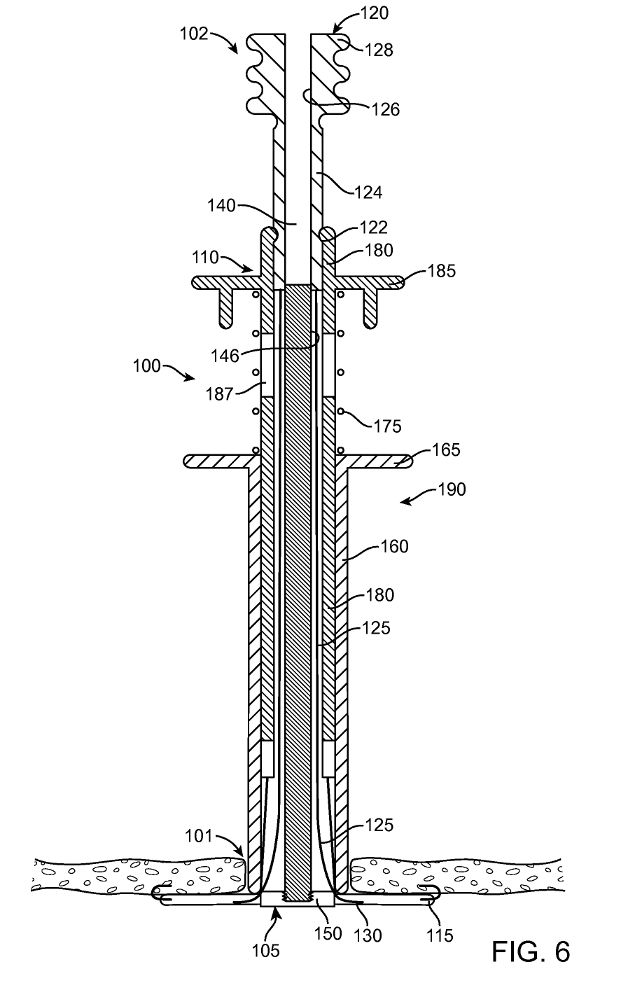

FIG. 6 illustrates a cross-sectional view of the wound closure device 100 in an engaged configuration, according to an example of the present disclosure. As depicted in FIG. 6, the engagement mechanism 120 of applicator 110 has been translated toward the proximal end of applicator 110 causing the first detent 122 to release the engagement mechanism 120 and allow the engagement mechanism 120 to further translate to engage the second detent 123. As a result of translation of the engagement mechanism 120 toward the proximal end 102 of the applicator 110, tension is applied to the plurality of tethers 125 coupled to the plurality of tissue engagement hooks 115, thereby causing the tissue engagement hooks 115 to move inward toward the implant main body 150 and engagingly penetrate the body tissue.

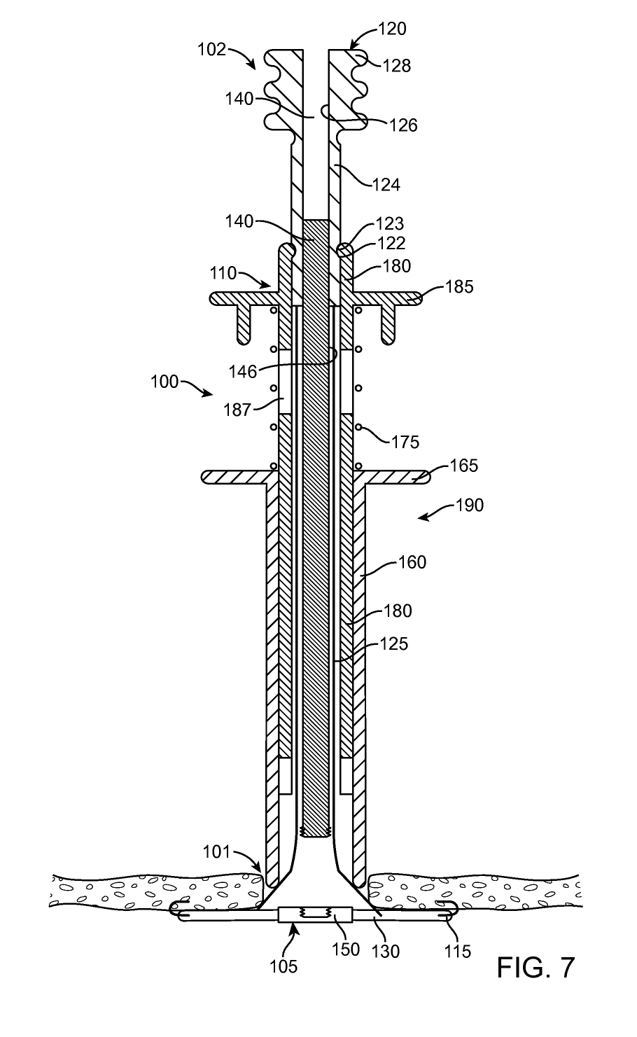

FIG. 7 illustrates a cross-sectional view of the wound closure device 100 in an engaged configuration with the pull rod 140 of the applicator 110 disengaged from the implant main body 150, according to an example of the present disclosure. As depicted in FIG. 7, the pull rod 140 has been unscrewed from the implant main body 150 by rotating the engagement mechanism 120, coupled to the pull rod 140, relative to the implant main body 150. In some cases, first detent 122 serves to prevent upward movement of the engagement mechanism 120 relative to the applicator main body 180 during rotation of the engagement mechanism 120. As shown in FIG. 7, upon unscrewing the pull rod 140 from the implant main body 150, the applicator 110 is disengaged from the implant main body 150 except for the coupling of the plurality of tethers 125 to the plurality of tissue engagement hooks 115.

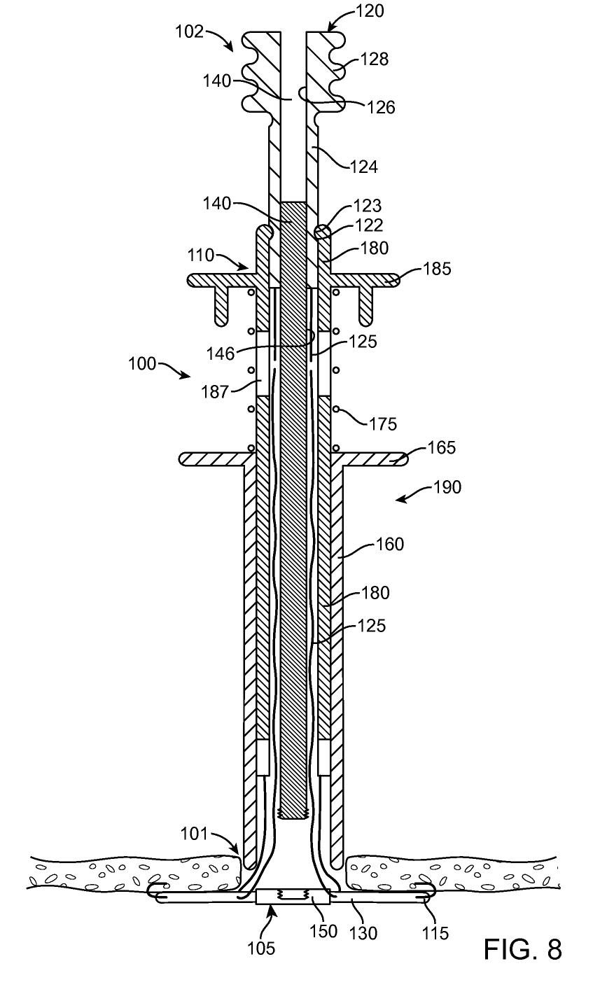

FIG. 8 illustrates a cross-sectional view of the wound closure device 100 in an engaged configuration with the plurality of tethers 125 severed and the applicator 110 disengaged from the implant main body 150, according to an example of the present disclosure. The plurality of tethers 125 can be severed by any suitable means, without departing from the spirit and scope of the present disclosure. For example, as depicted in FIG. 8, the plurality of tethers 125 can be accessed through aperture 187 in the applicator main body 180 and severed using surgical scissors, scalpel, or burning instrument. In some cases, a cutting surface built into the applicator can be used to sever the tethers. In such cases, the wound closure device 100 can be implemented without aperture 187 or aperture 187 can be used as a back-up mechanism for severing the tethers 125 in the event of failure of the primary cutting mechanism built into the applicator. In some cases, the plurality of tethers 125 can be formed in one or more loops such that severing the plurality of tethers accessed through a single aperture 187, on just one side of the applicator main body 180, is sufficient to cause severance of the plurality of tethers 125 so that the applicator 110 can be detached from the implant main body 150. In some cases, the plurality of tethers 125 can be formed in one or more loops such that severing a single tether 125 can result in severance of more than one of the plurality of tethers 125. Additionally, each tether 125 can be formed in a loop to form a loop tether. In at least one aspect of the present disclosure, the aperture 187 provides access to the plurality of tethers 125 only when the engagement mechanism 120 has been translated toward the proximal end 102 of the applicator 110. In some cases, aperture 187 only provides access to the plurality of tethers 125 when the engagement mechanism 120 has engaged the second detent 123.

The applicator 110 can also be configured such that disengagement of the applicator 110 from the implant main body 150 at the reversable attachment point simultaneously or automatically severs the plurality of tethers 125. For example, the applicator 110 can be configured such that unscrewing the pull rod 140 from the implant main body 150 causes simultaneous or automatic severance of the plurality of tethers 125, allowing for complete disengagement of the applicator 110 from the installed implant main body 150.

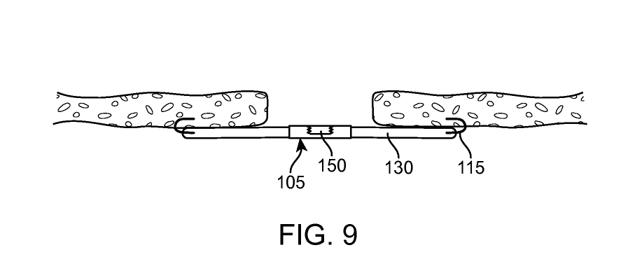

FIG. 9 illustrates a cross-sectional view of the implant 105 engaged with a layer of body tissue following disengagement and withdrawal of the applicator 110, according to an example of the present disclosure. As depicted in FIG. 9, the installed implant 105 includes a plurality of tissue engagement hooks 115 penetratingly engaged with at least one layer of body tissue. The plurality of tissue engagement hooks 115 are mounted within a respective one of the plurality of connecting members 130 which are coupled with the implant main body 150. As shown in FIG. 9, the implant 105, including tissue engagement hooks 115, connecting members 130 and implant main body 150 span and close the fascial wound.

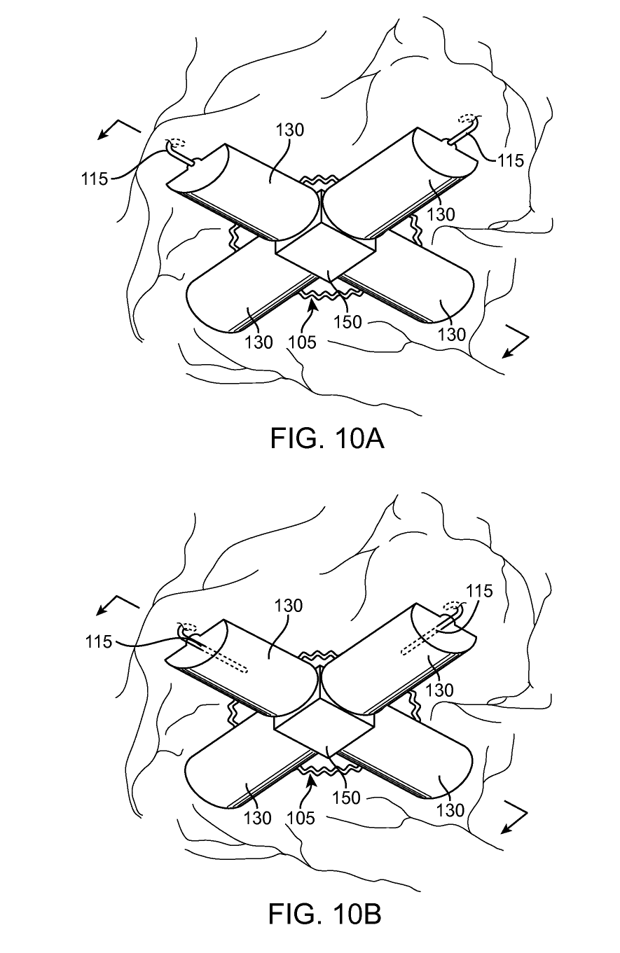

FIG. 10A illustrates a bottom perspective view of an implant 105 engaged with body tissue by a plurality of tissue engagement hooks 115, according to an example of the present disclosure. As depicted in FIG. 10A, a plurality of tissue engagement hooks 115 are in partial penetrating engagement with body tissue prior to tension being applied to the plurality of tethers 125 so as to translate the tissue engagement hooks 115 toward the implant main body 150.

FIG. 10B illustrates a bottom perspective view of an installed implant 105 engaged with body tissue by a plurality of tissue engagement hooks 115, according to an example of the present disclosure. As depicted in FIG. 10B, a plurality of tissue engagement hooks 115 are in penetrating engagement with body tissue following application of tension to a plurality of tethers 125 to translate the plurality of tissue engagement hooks toward the implant main body 150. The applicator (not shown) has been disengaged from the implant main body 150 and withdrawn leaving the fascial wound spanned and closed by implant 105 including implant main body 150 and connecting members 130 extending from implant main body 150.

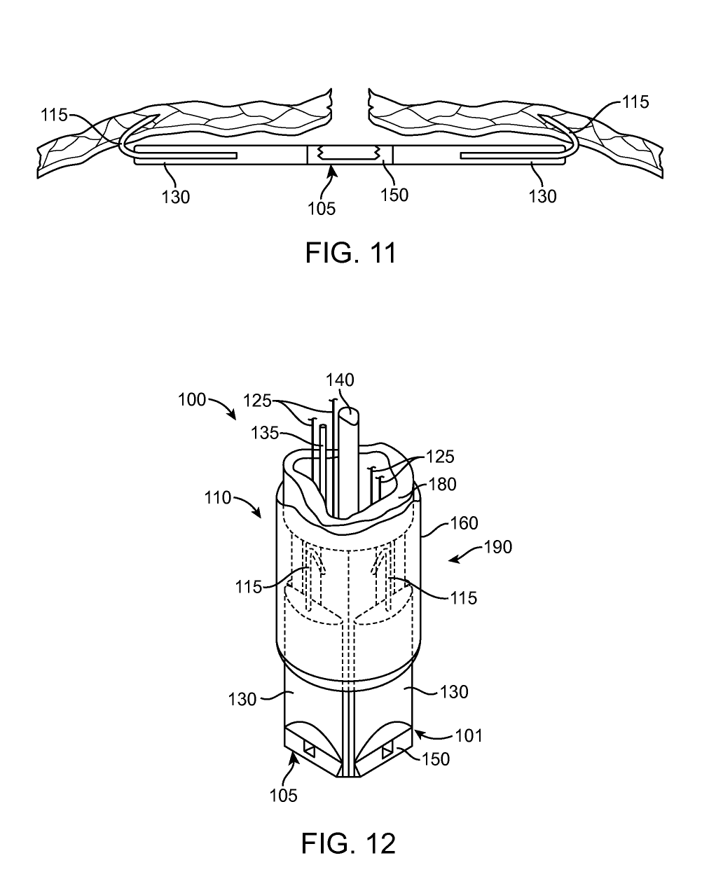

FIG. 11 illustrates a cross-sectional view of an implant 105 installed to close a fascial wound by a plurality of tissue engagement hooks 115, according to an example of the present disclosure. As depicted in FIG. 11, a plurality of tissue engagement hooks 115 are in penetrating engagement with body tissue after application of tension to a plurality of tethers 125 to translate the plurality of tissue engagement hooks toward the implant main body 150. The applicator (not shown) has been disengaged from the implant main body 150 and withdrawn leaving the fascial wound spanned and closed by implant 105 including implant main body 150 and connecting members 130 extending from implant main body 150.

FIG. 12 illustrates a perspective view of the distal portion 101 of a wound closure device 100 in the retracted configuration, according to an example of the present disclosure. As depicted in FIG. 12, the plurality of connecting members 130 and plurality of tissue engagement hooks 115 are in a retracted configuration suitable for insertion of the distal portion 101 of the wound closure device 100 into an abdominal cavity beyond a location of a fascial wound. The tissue engagement hooks 115 and a portion of the connecting members 130 are surrounded by securement member 190 in the form of a retractable sleeve 160. The plurality of tissue engagement hooks 115 are coupled to a plurality of tethers 125. The applicator 110 portion of the wound closure device 100 includes a push rod 140 that is releasably engaged with an implant main body 150.

The connecting members 130 are coupled with an extension assembly 135 which, upon actuation, is configured to extend the plurality of connecting members from the implant main body 150 by causing the connecting members 130 to rotate about the implant main body 150. The extension assembly 135 can include a linkage, such as a push wire, that is configured to extend the plurality of connecting members 130 upon actuation of the extension assembly 135.

FIG. 13A illustrates a cross-sectional view of the distal portion 101 of a wound closure device 100 with tissue engagement hooks 115 in the retracted configuration, according to an example of the present disclosure. As depicted in FIG. 13A, in the retracted configuration, the connecting member 130 is substantially parallel to the applicator main body 180 and pull rod 140. Further, in the retracted configuration, a securement member 190 at least partially surrounds connecting member 130 and tissue engagement hook 115. As depicted in FIG. 13A, the plurality of tissue engagement hooks 115 are closer to the proximal end 102 of the applicator 110 in the retracted configuration than in the engaged configuration.

FIG. 13B illustrates a cross-sectional view of the distal portion 101 of a wound closure device with tissue engagement hooks 115 in the engaged configuration and extended away from the implant main body 150, according to an example of the present disclosure. As depicted in FIG. 13B, securement member 190, in the form of retractable sleeve 160, has been retracted allowing for extension of connecting members 130 by actuation of extension assembly 135. As shown in FIG. 11, extension assembly 135 includes a linkage, such as a push wire, that upon actuation of the extension assembly 135, causes connecting member 130 to rotate about the implant main body 150 to extend from implant main body 150. The tissue engagement hook 115 is shown in FIG. 13B in the engaged configuration following upward motion of the wound closure device to bring the tissue engagement hook 115 into engagement with body tissue.

FIG. 13C illustrates a cross-sectional view of the distal portion 101 of a wound closure device 100 with tissue engagement hooks 115 in the engaged configuration and drawn toward the implant main body 150, according to an example of the present disclosure. As depicted in FIG. 13C, an engagement assembly has been activated to cause the tissue engagement hooks 115 to draw toward the implant main body 150 so as to penetrating engage the body tissue. As shown in FIG. 13C, activation of the engagement mechanism included application of tension to the plurality of tethers 125 so as to shift the tissue engagement hooks 115 toward the implant main body 150, thereby causing the tissue engagement hooks 115 to penetrating engage the body tissue.

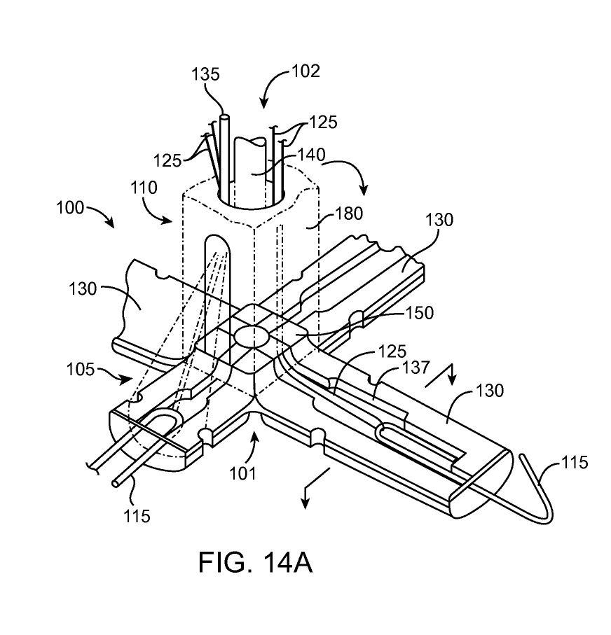

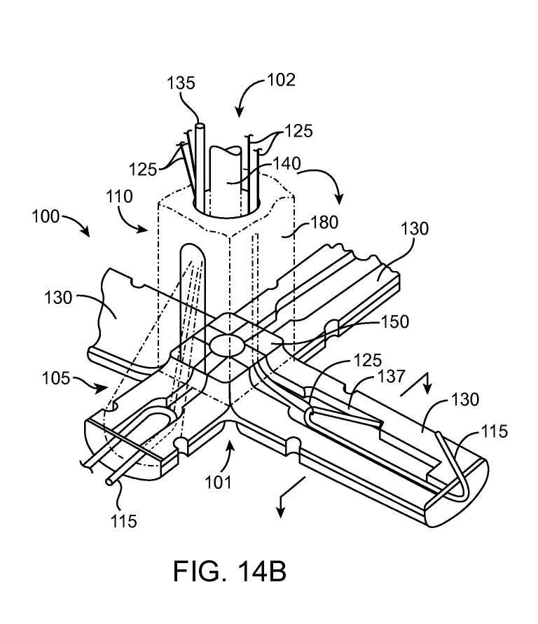

FIG. 14A illustrates a perspective view of the distal portion 101 of a wound closure device 100 having a spring deployment retainer 137, according to an example of the present disclosure. As depicted in FIG. 14A, the plurality of connecting members 130 are extended from implant main body 150. Each of the plurality of tissue engagement hooks 115 are coupled to applicator 110 by a tether 125. As shown in FIG. 14A, each tether 125 is formed into a tether loop that loops around the end of a corresponding tissue engagement hook 115. Application of tension to the plurality of tethers 125 causes each of the plurality of tissue engagement hooks 115 to translate toward the implant main body 150 from an extended position to an engaged position in which the tissue engagement hook 115 is in penetrating engagement with at least one layer of body tissue.

Each of the plurality of connecting members 130 includes a spring deployment retainer 137 configured to capture a proximal end of a corresponding tissue engagement hook 115 after the tissue engagement hook 115 is moved to an engaged configuration. As depicted in FIG. 14A, the spring deployment retainer 137 is in the form of a receiving space formed in each of the plurality of connecting members 130. The proximal end of each of the plurality of tissue engagement hooks is configured to expand upon being placed within the receiving space, thereby preventing the tissue engagement hook 115 from returning to the extended position. As a result, the spring deployment retainer 137 maintains the tissue engagement hook 115 in the engaged position.

FIG. 14B illustrates a perspective view of the distal portion 101 of a wound closure device 100 in the engaged configuration with the proximal end of a tissue engaging hook 115 captured in the receiving space of a spring deployment retainer 137, according to an example of the present disclosure. As depicted in FIG. 14B, the proximal end of each of the plurality of tissue engagement hooks 115 enters the receiving space of the spring deployment retainer 137 upon application of tension to the plurality of tethers 125 so that the proximal end of the tissue engagement hook 115 is captured by a portion of the receiving space thereby preventing further translational movement of the corresponding tissue engagement hook 115.

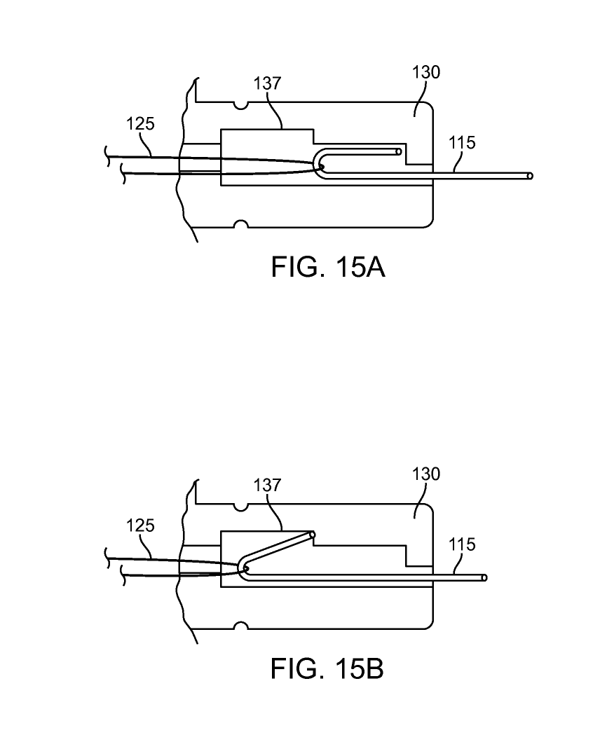

FIG. 15A illustrates a close-up cross-sectional view of the spring deployment retainer depicted in FIGS. 14A and 14B, according to an example of the present disclosure. As depicted in FIG. 15A, the proximal end of tissue engagement hook 115 is mounted within connecting member 130 and coupled with tether 125. As shown in FIG. 15B, upon application of tension to tether 125, the tissue engagement hook 115 translates toward the implant main body 150 until the proximal end of the tissue engagement hook 115 is captured by a portion of the receiving space of the spring deployment retainer 137, thereby preventing further translational movement of the corresponding tissue engagement hook 115. The spring deployment retainer 137 maintains the tissue engagement hook 115 in the engaged position so as to maintain the tissue engagement hook 115 in penetrating engagement with the body tissue.

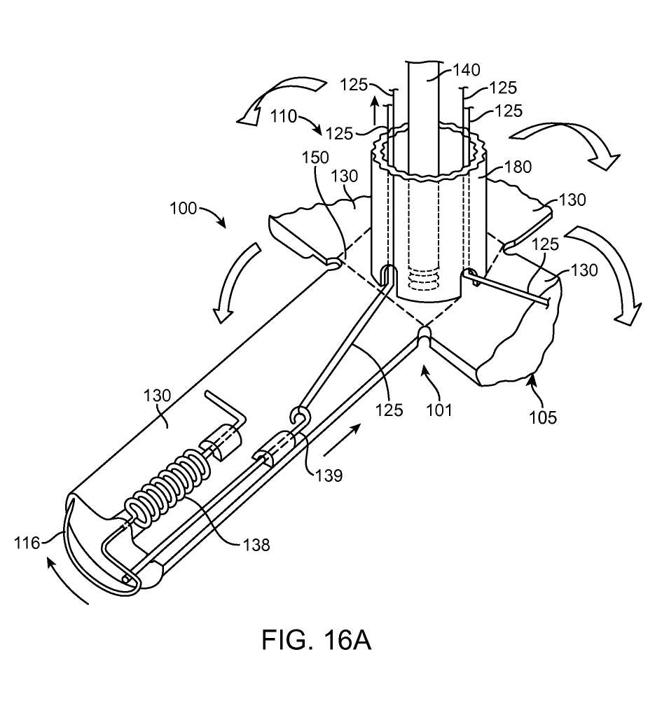

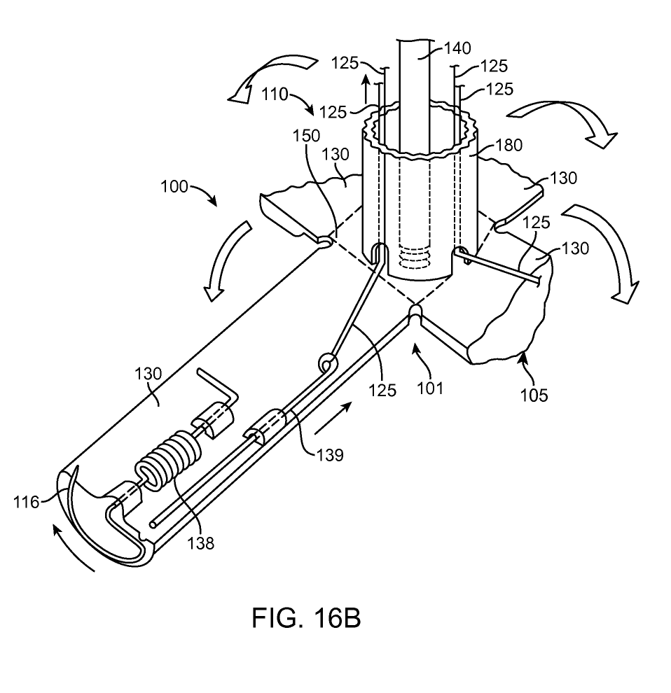

FIG. 16A is a perspective view of the distal portion 101 of a wound closure device 100 having rotating tissue engagement hooks 116 held in an unactuated condition by a retention device, according to an example of the present disclosure. As depicted in FIG. 16A, the connecting members 130 are extended from implant main body 150 which is releasably coupled with pull rod 140 disposed within applicator main body 180. Each of the plurality of rotating tissue engagement hooks 116 are coupled with a biasing member 138 held in an unactuated condition by a retention device 139 coupled with a tether 125. As shown in FIG. 16A, the biasing member 138 is a coil torsion spring, the tether 125 is formed into a tether loop and the retention device 139 is a lock-out pin. However, any type of biasing member can be utilized in the wound closure device 100 without departing from the spirit and scope of the present disclosure. For example, the biasing member can be an elastically deformed material. In one example, the biasing member can be a steel. Further, any mechanism capable of maintaining the biasing member in an unactuated position can be utilized in the wound closure device 100 without departing from the spirit and scope of the present disclosure. Additionally, any mechanism for disengaging the retention device may be utilized in the wound closure device 100 without departing from the spirit and scope of the present disclosure.

As shown in FIG. 16B, application of tension to the tether 125 causes the tether 125 to translate the retention device 139 from an extended unactuated condition to a retracted engagement position, thereby disengaging the retention device 139 and allowing the biasing member 138 to cause the rotation of the rotating tissue engagement hook 116. The rotating tissue engagement hook 116, upon rotation, is configured to penetrate at least one layer of body tissue.

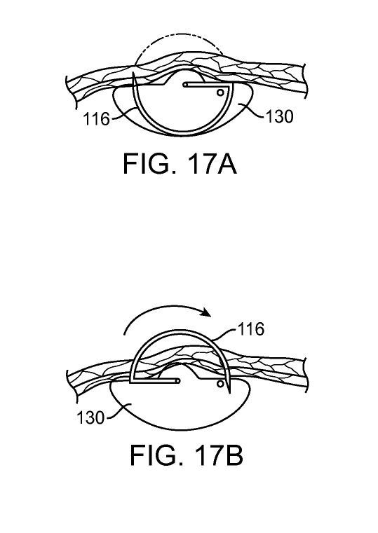

FIG. 17A illustrates a close-up cross-sectional view of a rotating tissue engagement hook 116 in the unactuated condition, according to an example of the present disclosure. As depicted in FIG. 17A, the rotating tissue engagement hook 116 is in an unactuated condition as maintained by retention device (not shown). FIG. 17B illustrates a close-up cross-sectional view of a rotating tissue engagement hook 116 in the engaged position, according to an example of the present disclosure. As depicted in FIG. 17B, the rotating tissue engagement hook 116 has penetrated both sides of at least one layer of body tissue by rotating upon disengagement of the retention device (not shown).

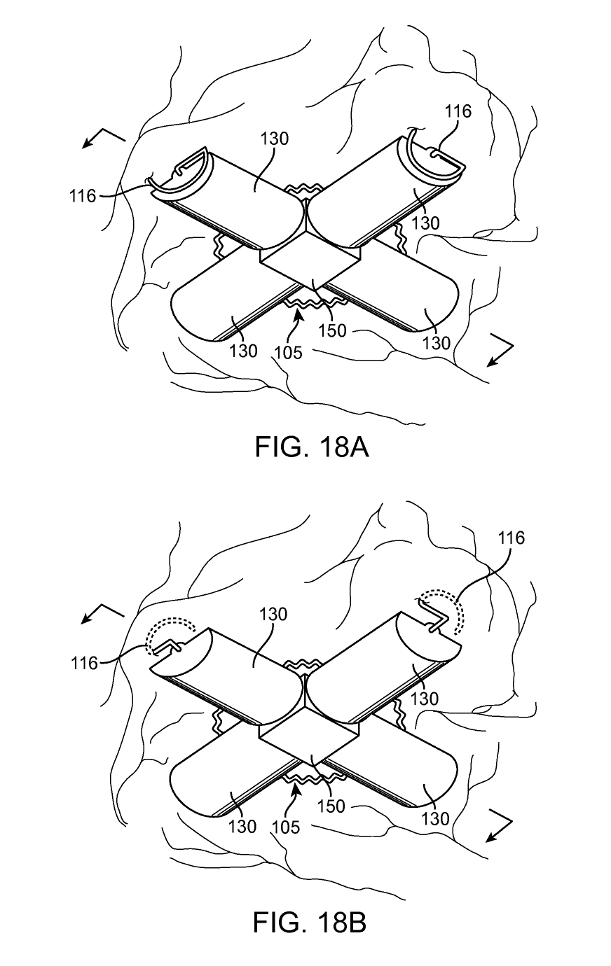

FIG. 18A illustrates a bottom perspective view of an implant 105 having a plurality of rotating tissue engagement hooks 116 in pressure contact with body tissue, according to an example of the present disclosure. As depicted in FIG. 18A, the rotating tissue engagement hooks 116 are held in an unactuated condition by retention device (not shown). FIG. 18B is a bottom perspective view of an installed implant 105 engaged with body tissue by a plurality of rotating tissue engagement hooks 116, according to an example of the present disclosure. As depicted in FIG. 18B a plurality of rotating tissue engagement hooks 116 are in penetrating engagement with body tissue after disengagement of retention device 139, by application of tension to a plurality of tethers 125, thereby causing the rotating tissue engagement hooks 116 to rotate and penetrate the body tissue. The applicator (not shown) has been disengaged from the implant main body 150 and withdrawn leaving the fascial wound spanned and closed by implant 105 including implant main body 150 and connecting members 130 extending from implant main body 150.

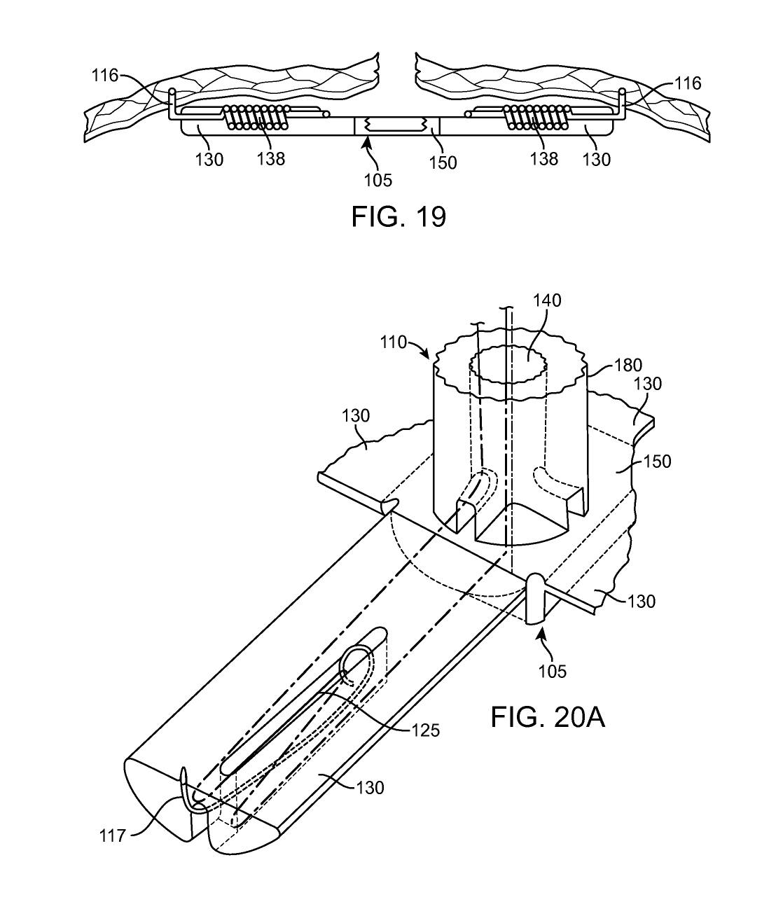

FIG. 19 illustrates a cross-sectional view of an implant 105 installed to close a fascial wound by a plurality of rotating tissue engagement hooks 116, according to an example of the present disclosure. As depicted in FIG. 19, a plurality of rotating tissue engagement hooks 116 are in penetrating engagement with body tissue after rotation caused by a corresponding plurality of biasing members 138. The applicator (not shown) has been disengaged from the implant main body 150 and withdrawn leaving the fascial wound spanned and closed by implant 105 including implant main body 150 and connecting members 130 extending from implant main body 150.

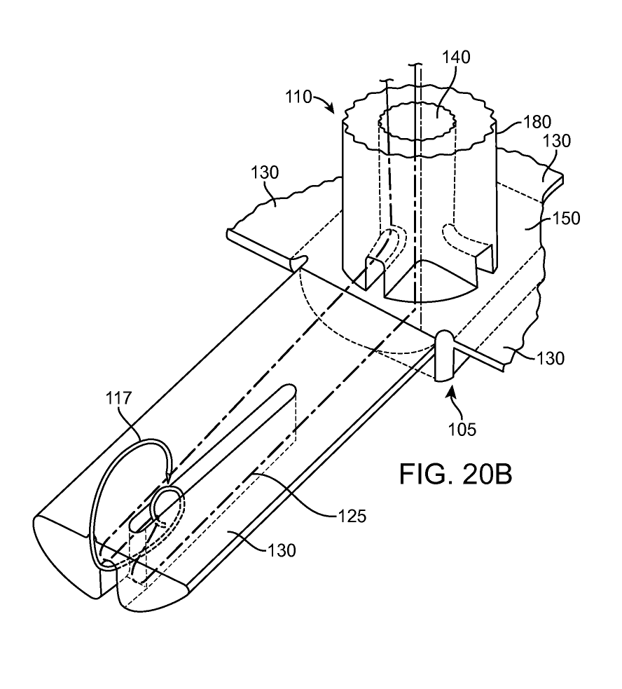

FIG. 20A illustrates a perspective view of the distal portion of a wound closure device having pre-curled tissue engagement hooks 117 in a retracted and biased state, according to an example of the present disclosure. As depicted in FIG. 20A, connecting members 130 are extended from implant main body 150 releasably coupled with pull rod 140 disposed within applicator main body 180. Pre-curled tissue engagement hooks 117 are shown in FIG. 20A in a retracted and biased state. Each pre-curled tissue engagement hook 117 is elastically deformed within an interior portion of the connecting member 130 and slidably disposed about the connecting member 130. Additionally, each pre-curled tissue engagement hook 117 is coupled with a corresponding tether 125. The pre-curled tissue engagement hooks 117 are configured to penetrate at least one layer of body tissue upon application of tension to the tether 125 thereby causing the pre-curled tissue engagement hook 117 to slide outward from within the interior portion of the connecting member 130. As compared to the pre-curled tissue engagement hooks 117 in the deployed and unbiased state, the retracted and biased pre-curled tissue engagement hooks 117 have less of a curl or no curl.

FIG. 20B illustrates the pre-curled tissue engagement hooks 117 in a deployed and unbiased curled state after application of tension to the tether 125 caused the pre-curled hook 117 to slide outward from within the interior portion of the connecting member 130. As shown in FIGS. 20A and 20B, the tether 125 is formed into a tether loop that loops around a corresponding pre-curled hook 117.

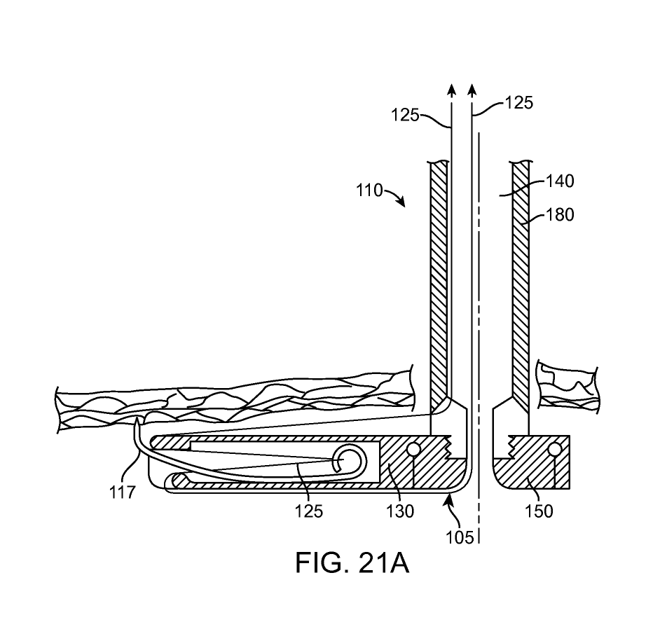

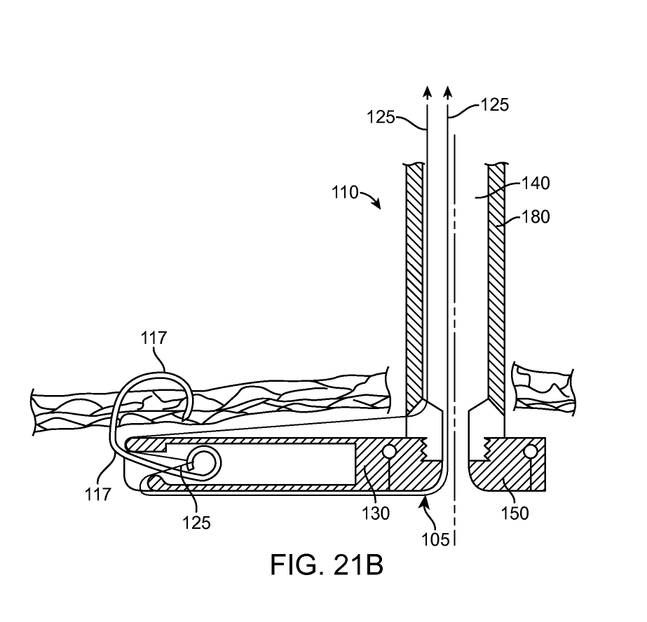

FIG. 21A illustrates a cross-sectional view of the distal portion of a wound closure device having pre-curled tissue engagement hooks 117 in a retracted and biased state, according to an example of the present disclosure. As depicted in FIG. 21A, connecting members 130 are extended from implant main body 150 releasably coupled with pull rod 140 disposed within applicator main body 180. The pre-curled tissue engagement hooks 117 are shown in FIG. 21A in a retracted and biased state. Each pre-curled tissue engagement hook 117 is elastically deformed within an interior portion of the connecting member 130 and slidably disposed about the connecting member 130. Additionally, each pre-curled tissue engagement hook 117 is coupled with a corresponding tether 125. The pre-curled tissue engagement hooks 117 are configured to penetrate at least one layer of body tissue upon application of tension to the tether 125 thereby causing the pre-curled tissue engagement hook 117 to slide outward from within the interior portion of the connecting member 130. As shown in FIG. 21A, the pre-curled tissue engagement hook 117 is in pressure contact with a layer of body tissue.

FIG. 21B illustrates the pre-curled tissue engagement hooks 117 in penetrating engagement with at least one layer of body tissue following application of tension to the tether 125, causing the pre-curled tissue engagement hook 117 to slide outward from the implant main body 150 and from within an interior portion of the connecting member 130. As shown in FIG. 21B, the tether 125 is formed into a loop coupled to an engagement mechanism on the applicator (not shown).

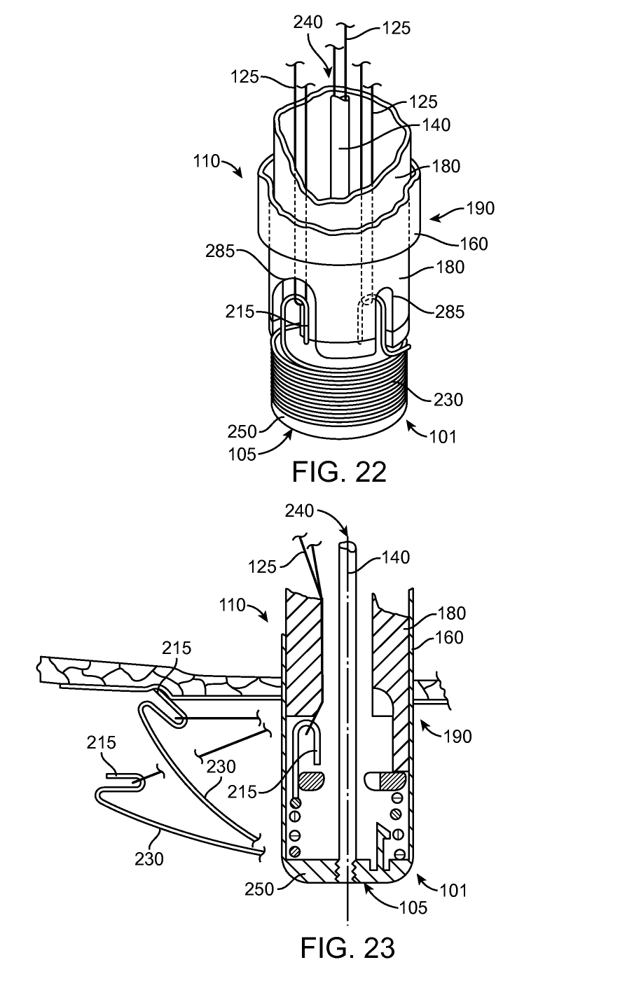

FIG. 22 illustrates a perspective view of the distal portion 101 of a wound closure device having elastic members 230 with a tissue engagement hook 215 formed at the end, according to an example of the present disclosure. As depicted in FIG. 22, the wound closure device includes a plurality of elastic members 230 coupled to the implant main body 250. Each of the plurality of elastic members 230 has a tissue engagement hook 215 formed on the distal end. In some cases, the plurality of elastic members 230 can be nested wire arms. In some cases, there can be four elastic members 230, each with a tissue engagement hook 215 formed at the end.

In a first configuration, the elastic members 230 are at least partially wound around the applicator main body 180 and/or the implant main body 250. In the first configuration, the plurality of tissue engagement hooks 215, as well as the plurality of elastic members 230, are retained by a securement member 190 having a retractable sleeve 160. The securement member 190 is coupled to applicator 110 having a distal end 101 configured to releasably engage implant main body 250. As depicted in FIG. 22, the applicator 110 includes pull rod 140 releasably attached to implant main body 250 by a screw mechanism. However, any mechanism for releasably attaching the applicator 110 to the main implant body 250 can be utilized in the wound closure device without departing from the spirit and scope of the present disclosure.

The wound closure device further includes tethers 125 coupled to corresponding ones of the plurality of tissue engagement hooks 215. As depicted in FIG. 22, the tethers 125 are formed into tether loops. In some cases, the plurality of tissue engagement hooks 215 can have a U-shaped portion such that the distal end of the plurality of tissue engagement hooks 215 is configured to contact the tissue first. In such cases, the U-shaped portion of the tissue engagement hooks 215 can be configured to receive a corresponding one of the one or more tethers 125.

As shown in FIG. 22, the applicator main body 180 has a plurality of apertures 285 formed therethrough such that each of the plurality of apertures 285 correspond to one of the plurality of tissue engagement hooks 215. In some cases, the plurality of apertures 285 can be a substantially upside down U-shaped aperture, as depicted in FIG. 22. However, the apertures 285 can be any suitable shape without departing from the spirit and scope of the present disclosure. In the first configuration, the plurality of tissue engagement hooks 215 can be retained within the plurality of apertures 285. However, the plurality of tissue engagement hooks 215 can be retained in any suitable structure in the first configuration without departing from the spirit and scope of the present disclosure.

In a second configuration, the plurality of elastic members 230 are biased outward from the implant main body 250 following retraction of the retractable sleeve 160, such that the plurality of tissue engagement hooks 215 are located away from the implant main body 250. In the first configuration, the plurality of tissue engagement hooks 215 are closer to the implant main body 250 than in the second configuration.

FIG. 23 illustrates a cross-sectional view of the distal portion 101 of a wound closure device having elastic members 230 with a tissue engagement hook formed at the end, according to an example of the present disclosure. As depicted in FIG. 23, the tethers 125 are configured to adjust deployment of the plurality of tissue engagement hooks 215 so that from the second configuration the hooks can be returned to be closer to the applicator 110. Further, the tethers 125 are configured to bring the plurality of tissue engagement hooks 215 into a suitable position and orientation for tissue penetration. In some cases, the tethers 125 can be configured to bring the plurality of tissue engagement hooks 215 into pressure contact with a layer of body tissue while in the second configuration.

The tissue engagement hooks 215 can be brought into pressure contact with a layer of body tissue by applying an upward pulling motion on the applicator 110. The wound closure device can further include an engagement mechanism 240 capable of bringing the plurality of tissue engagement hooks 215 into pressure contact with a layer body tissue when the wound closure device is in the second configuration. In some cases, the engagement mechanism 240 can be coupled to the implant main body 250. The engagement mechanism 240 can include a pull rod 140 in some cases. In some cases, the engagement mechanism 240 can have a distal end that is threaded and configured to threadingly engage with the implant main body 250, so that the pull rod 140 can be disengaged from the implant main body 250 once the implant 105 is installed in the patient.

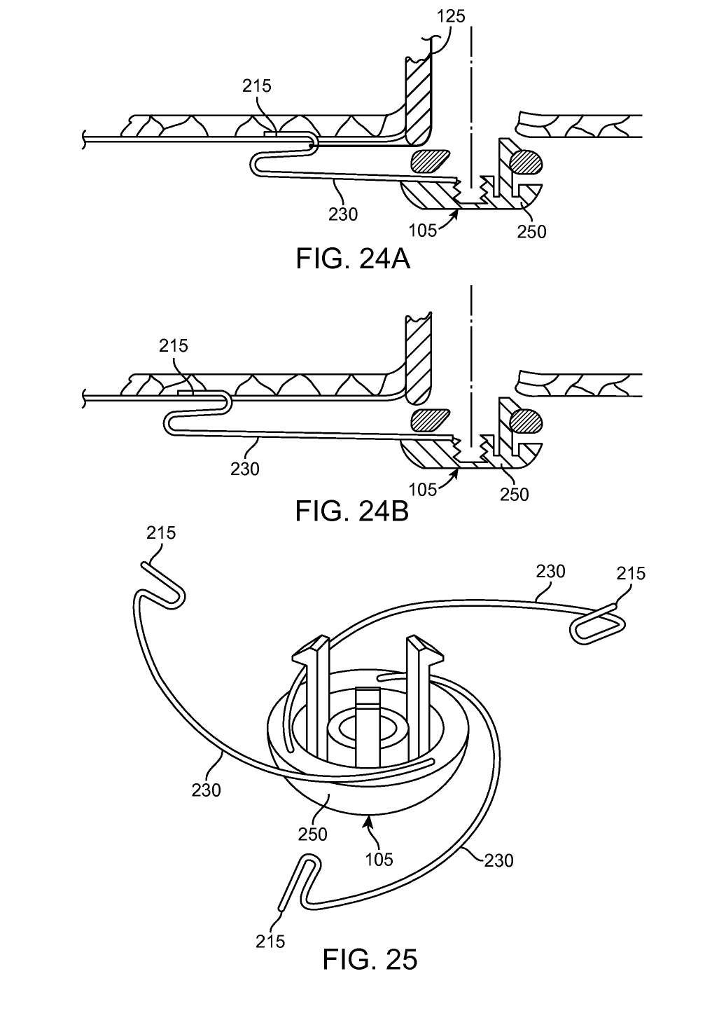

Either application of an upward motion pulling motion on applicator 110 or actuation of engagement mechanism 240 can also bring the tissue engagement hooks 215 and elastic members 230 into a flatter or more horizontal orientation that can constitute a more advantageous tissue penetration angle for tissue engagement, as shown in FIG. 24A. As depicted in FIG. 24A, the plurality of tissue engagement hooks 215 are still coupled to tether 125. As depicted in FIG. 24B, the tethers 125 can be released to cause the plurality of tissue engagement hooks 215 to elastically extend away further away from the implant main body 250 so as to increasingly penetrate and engage the tissue. In some cases, the tethers 125 can be released causing tissue engagement by the tissue engagement hooks 215 prior to application of an upward motion pulling motion on applicator 110 or actuation of engagement mechanism 240 to bring tissue engagement hooks 215 into a flatter or more horizontal orientation.

FIG. 25 illustrates a perspective view of the implant main body 250 coupled to a plurality of elastic members 230 capable of elastically extending away from the implant main body 250 upon retraction of the securement member 190 to form the second configuration. In some cases, the implant main body 250 can include an anchor ring.

According to the present disclosure, a wound closure device is provided. The wound closure device includes an applicator having a distal end and a proximal end. The distal end of the applicator is configured to releasably engage an implant main body. The implant main body is coupled with a plurality of connecting members configured to extend from the implant main body. The wound closure device further includes a plurality of tissue engagement hooks. Each of the plurality of tissue engagement hooks are mounted within a respective one of the plurality of connecting members. The plurality of connecting members are configured to rotate about the implant main body, so that in a retracted configuration the plurality of tissue engagement hooks are closer to the proximal end of the applicator than in an engaged configuration.

According to at least one aspect of the present disclosure, the applicator includes a securement member configured to at least partially surround the plurality of tissue engagement hooks. In some cases, the securement member can be a retractable sleeve. In some cases, the securement member or the retractable sleeve, upon retraction, can be configured to at least partially extend the plurality of connecting members from the implant main body. In some cases, the securement member or the retractable sleeve is further configured to engage the plurality of partially extended connecting members to cause the plurality of connecting members to further extend from the implant main body.

In at least one aspect of the present disclosure, the applicator is configured to bring the plurality of tissue engagement hooks into pressure contact with at least one layer of tissue upon application of a pulling motion to the applicator. In at least one aspect of the present disclosure, the applicator includes an engagement mechanism configured to bring the plurality of tissue engagement hooks into pressure contact with at least one layer of tissue upon actuation of the engagement mechanism. In some cases, the engagement mechanism can include a plunger. In some cases, the plunger can include a ring collar. In some cases the engagement mechanism can include a pull rod. The engagement mechanism can also include a hook activation member configured to bring the plurality of tissue engagement hooks into pressure contact with at least one layer of tissue upon application of a pulling motion on the hook activation member. In some cases, the hook activation member can be a ring collar. In some cases, the engagement mechanism can further include a pull rod coupled to the hook activation member that is configured to translate relative to the pull rod and be rotationally connected to the pull rod.

In some cases, the engagement mechanism can include a plunger and a pull rod. The plunger can include an aperture having a surface configured to matingly engage an external surface of the pull rod such that rotation of the plunger relative to the implant main body causes rotation and release of the pull rod from the implant main body. In some cases, the aperture surface of the plunger and the external surface of the pull rod are each hexagonal. The plunger can further include a ring collar.

In at least one aspect of the present disclosure, the applicator includes an engagement mechanism configured to cause the plurality of tissue engagement hooks to penetrate at least one layer of body tissue upon actuation of the engagement mechanism. In some cases, the engagement mechanism includes a plurality of tethers each coupled with a corresponding one of the plurality of tissue engagement hooks. In some cases, each of the plurality of tethers is formed into a loop. In some cases, the plurality of tethers are configured to cause the plurality of tissue engagement hooks to penetrate at least one layer of body tissue upon the application of tension to the plurality of tethers.

According to at least one aspect of the present disclosure, the engagement mechanism includes a plunger and a detent. The plunger is configured to apply tension to the plurality of tethers upon release of the detent from engagement with the applicator main body. The plunger can include a ring collar in some cases. In some cases, the plurality of tethers can be configured to be severed upon rotation of the ring collar relative to the implant main body.

In at least one aspect of the present disclosure, the applicator can further include one or more apertures configured to provide sufficient access to the plurality of tethers so that the plurality of tethers can be severed. When the plurality of tethers are formed into one or more loops, a single applicator aperture can provide sufficient access to the plurality of tethers so that the plurality of tethers can be severed without accessing the tethers on the opposite side of the applicator.

In at least one aspect of the present disclosure, the applicator can include a pull rod configured to cause the release of the applicator from the implant main body upon rotation of the pull rod relative to the implant main body. In some cases, the plurality of tethers can be configured to be severed upon rotation of the pull rod relative to the implant main body.

In at least one aspect of the present disclosure, the engagement mechanism can be configured to cause the release of the applicator from the implant main body upon mechanical release of the engagement mechanism, or a portion thereof, relative to the implant main body. In some cases, the engagement mechanism can be configured to mechanically release upon rotation of the engagement mechanism, or a portion thereof, relative to the implant main body.

In at least one aspect of the present disclosure, the applicator includes an extension assembly configured, upon actuation, to extend the plurality of connecting members from the implant main body. In some cases, the extension assembly can include a linkage which, upon actuation, is configured to extend the plurality of connecting members from the implant main body. In certain cases, the linkage can be a push wire.

In at least one aspect of the present disclosure, the plurality of connecting members each includes a spring deployment retainer configured to capture a proximal end of a corresponding one of the plurality of tissue engagement hooks after being moved to an engaged configuration. The spring deployment retainer can be in the form of a receiving space formed in the corresponding one of the plurality of connecting members, and the proximal end of the one of the plurality of tissue engagement hooks can be configured to expand upon being placed within the receiving space, thereby preventing the hook to return to a state prior to the engaged configuration. In some cases, the proximal end of the one of the plurality of tissue engagement hooks enters the receiving space upon application of tension to the plurality of tethers, whereby the proximal end of the one of the plurality of tissue engagement hooks is captured by a portion of the receiving space that prevents translational movement of the corresponding tissue engagement hook.

In at least one aspect of the present disclosure, the plurality of tissue engagement hooks each includes one or more rotating hooks coupled with a biasing member, the one or more rotating hooks can be held in an unactuated condition by a retention device coupled with a tether. The rotating hook is configured to penetrate at least one layer of body tissue upon application of tension to the tether thereby disengaging the retention device and allowing the biasing member to cause the rotation of the corresponding hook to engage the tissue. In some cases, the biasing member can be a torsion spring. The retention device can be a lock-out pin in some cases.

In at least one aspect of the present disclosure, the plurality of tissue engagement hooks each comprise a pre-curled hook, which in an deployed and unbiased state is curled and in a retracted and biased state is deformed such that the curled portion has less of a curl or no curl. Each pre-curled hook can be elastically deformed within an interior portion of the connecting member, slidably disposed about the connecting member and coupled with a corresponding one of the plurality of tethers. Each pre-curled hook can be configured to penetrate at least one layer of body tissue upon application of tension to the tether, thereby causing the pre-curled hook to slide outward from within the interior portion of the connecting member.

According to the present disclosure, a method for closing a fascial wound on a patient is provided. The method includes inserting a wound closure device into an abdominal cavity at least partially beyond a location of the fascial wound. The wound closure device includes an applicator releasably engaged with an implant main body, a plurality of connecting members, each configured to extend from the implant main body, and a plurality of tissue engagement hooks coupled with a respective one of the plurality of connecting members.

The method further includes extending the plurality of connecting members from the implant main body such that the connecting members rotate about the implant main body. The method also includes bringing the plurality of tissue engagement hooks into pressure contact with at least one layer of tissue and causing the plurality of tissue engagement hooks to penetrate at least one layer of body tissue. Finally, the method includes disengaging the applicator from the implant main body.

In at least one aspect of the present disclosure, the wound closure device is configured such that during insertion into the abdominal cavity the plurality of tissue engagement hooks are closer to a proximal end of the applicator than when engaged with the at least one layer of tissue.

In at least one aspect of the present disclosure, the method includes extending the plurality of connecting members from the implant main body by actuating the applicator. In some cases, extending the plurality of connecting members from the implant main body includes retracting a securement member at least partially surrounding the plurality of connecting members. In some case, extending the plurality of connecting members from the implant main body includes retracting a retractable sleeve. In some cases, extending the plurality of connecting members from the implant main body includes translating a retractable sleeve to engage the plurality of connecting members. In some cases, extending the plurality of connecting members from the implant main body comprises applying a deployment motion to a linkage of the applicator.

In at least one aspect of the present disclosure, bringing the plurality of tissue engagement hooks into pressure contact with at least one layer of tissue includes applying a pulling motion to the applicator. In some cases, bringing the plurality of tissue engagement hooks into pressure contact with at least one layer of tissue includes activating an engagement mechanism on the applicator. In some cases, bringing the plurality of tissue engagement hooks into pressure contact with at least one layer of tissue includes retracting the applicator. In some cases, bringing the plurality of tissue engagement hooks into pressure contact with at least one layer of tissue includes applying a pulling motion on a ring collar on the applicator. In some cases, bringing the plurality of tissue engagement hooks into pressure contact with at least one layer of tissue includes applying tension to a plurality of tethers coupled to a corresponding one of the plurality of tissue engagement hooks.

In at least one aspect of the present disclosure, causing the plurality of tissue engagement hooks to penetrate at least one layer of body tissue includes applying a pulling motion to the applicator. In some cases, causing the plurality of tissue engagement hooks to penetrate at least one layer of body tissue includes activating an engagement mechanism on the applicator. In some cases, causing the plurality of tissue engagement hooks to penetrate at least one layer of body tissue includes retracting the applicator. In some cases, causing the plurality of tissue engagement hooks to penetrate at least one layer of body tissue includes applying tension to a plurality of tethers coupled to a corresponding one of the plurality of tissue engagement hooks.

In at least one aspect of the present disclosure, applying tension to a plurality of tethers includes pulling a ring collar on the applicator until a detent releases the ring collar.

In at least one aspect of the present disclosure, disengaging the applicator from the implant main body includes rotating the applicator relative to the implant main body. In some cases, disengaging the applicator from the implant main body includes rotating a ring collar on the applicator relative to the implant main body. In some cases, disengaging the applicator from the implant main body includes rotating a pull rod on the applicator relative to the implant main body.

In at least one aspect of the present disclosure, the method further includes severing a plurality of tethers coupled with the plurality of tissue engagement hooks. In some cases, the severing of a plurality of tether occurs upon disengagement of the applicator from the implant body. In some cases, the severing of a plurality of tethers occurs upon rotation of a pull rod on the applicator relative to the implant main body. In some cases, severing a plurality of tethers occurs upon rotation of a ring collar on the applicator relative to the implant main body.

In at least one aspect of the present disclosure, disengaging the applicator from the implant main body includes severing a plurality of tethers coupled with the plurality of tissue engagement hooks. In some cases, disengaging the applicator from the implant main body includes activating a disengagement mechanism on the applicator that simultaneously or sequentially severs one or more tethers and mechanically releases the applicator from the implant main body. In some cases, activating a disengagement mechanism on the applicator includes rotating a pull rod on the applicator relative to the implant main body. In some cases, activating a disengagement mechanism on the applicator includes pulling a plunger on the applicator. In some cases, disengaging the applicator from the implant main body includes activating a disengagement mechanism on the applicator that simultaneously or sequentially severs one or more tethers and rotates a pull rod on the applicator to mechanically release the applicator from the implant main body.

According to the present disclosure, a wound closure device is provided that includes an applicator having a distal end and a proximal end. The distal end of the applicator is configured to releasably engage an implant main body. The wound closure device further includes a securement member coupled to the applicator and having a retractable sleeve. The wound closure device further includes a plurality of elastic members coupled to the implant main body. Each of the plurality of elastic members have a tissue engagement hook formed at a distal end. The wound closure device further includes a first configuration in which the plurality of tissue engagement hooks are retained by the retractable sleeve and are closer to the implant main body than in a second configuration in which the plurality of elastic members are biased outward from the implant main body, so that the plurality of tissue engagement hooks are located away from the implant main body.

In at least one aspect of the present disclosure, the wound closure device also includes one or more tethers coupled to corresponding ones of the plurality of tissue engagement hooks. The one or more tethers can be configured to adjust deployment of the plurality of tissue engagement hooks so that from the second configuration the hooks can be returned to be closer to the actuator. In some cases, the plurality of tissue engagement hooks can have a U-shaped portion such that the distal end of the plurality of tissue engagement hooks is configured to contact the tissue first. In some cases, the U-shaped portion can be configured to receive a corresponding one of the one or more tethers. In some cases, the one or more tethers can be a loop of material.

In at least one aspect of the present disclosure, the plurality of elastic members can be wound around the implant main body in the first configuration. In some cases, at least one of the plurality of elastic members can be partially wound around the retractable sleeve in the first configuration. In some cases, the plurality of elastic members number four and the plurality of tissue engagement hooks number four.

In at least one aspect of the present disclosure, the retractable sleeve has a plurality of apertures formed therethrough, such that each of the plurality of apertures corresponds to one of the plurality of tissue engagement hooks. In some cases, the plurality of apertures can be substantially an upside down U-shaped aperture.

In at least one aspect of the present disclosure, the wound closure device can include an engagement mechanism that is coupled to the implant main body. In some case, the engagement mechanism can be a pull rod. The engagement mechanism can also have a distal end that is threaded and configured to threadingly engage with the implant main body, so that the pull rod can be disengaged from the implant main body once the implant main body is installed in the patient.

### Statements of the Disclosure Include:

Statement 1: A wound closure device comprising an applicator having a distal end and a proximal end wherein the distal end is configured to releasably engage an implant main body; a plurality of connecting members, each configured to extend from the implant main body; a plurality of tissue engagement hooks, each of the plurality of tissue engagement hooks being mounted within a respective one of the plurality of connecting members; wherein the plurality of connecting members rotate about the implant main body, so that in a retracted configuration the plurality of tissue engagement hooks are closer to the proximal end of the applicator than in an engaged configuration.

Statement 2: A wound closure device according to Statement 1, wherein the applicator comprises a securement member configured to at least partially surround the plurality of tissue engagement hooks.

Statement 3: A wound closure device according to Statement 2, wherein the securement member is a retractable sleeve.

Statement 4: A wound closure device according to Statement 3, wherein the retractable sleeve, upon retraction, is configured to at least partially extend the plurality of connecting members from the implant main body.

Statement 5: A wound closure device according to Statement 3 or Statement 4, wherein the retractable sleeve is further configured to engage the plurality of partially extended connecting members to cause the plurality of connecting members to further extend from the implant main body.

Statement 6: A wound closure device according to Statement 2, wherein the securement member, upon retraction, is configured to at least partially extend the plurality of connecting members from the implant main body.

Statement 7: A wound closure device according to Statement 2 or Statement 6, wherein the securement member is further configured to engage the plurality of partially extended connecting members to cause the plurality of connecting members to further extend from the implant main body.

Statement 8: A wound closure device according to any one of the preceding Statements 1-7, wherein the applicator is configured to bring the plurality of tissue engagement hooks into pressure contact with at least one layer of tissue upon application of a pulling motion to the applicator.

Statement 9: A wound closure device according to any one of the preceding Statements 1-8, wherein the applicator comprises an engagement mechanism, wherein the engagement mechanism is configured to bring the plurality of tissue engagement hooks into pressure contact with at least one layer of tissue.

Statement 10: A wound closure device according to Statement 9, wherein the engagement mechanism comprises a plunger.

Statement 11: A wound closure device according to Statement 10, wherein the plunger comprises a ring collar.

Statement 12: A wound closure device according to any one of the preceding Statements 9-11, wherein the engagement mechanism comprises a pull rod.

Statement 13: A wound closure device according to any one of the preceding Statements 9-12, wherein the engagement mechanism comprises a hook activation member, wherein the hook activation member is configured to bring the plurality of tissue engagement hooks into pressure contact with at least one layer of tissue upon application of a pulling motion on the hook activation member.

Statement 14: A wound closure device according to Statement 13, wherein the hook activation member comprises a ring collar.

Statement 15: A wound closure device according to Statement 13 or Statement 14, wherein the engagement mechanism further comprises a pull rod coupled to the hook activation member, wherein the hook activation member is configured to translate relative to the pull rod and be rotationally connected to the pull rod.

Statement 16: A wound closure device according to Statement 9, wherein the engagement mechanism comprises a plunger and a pull rod, wherein the plunger includes an aperture having a surface configured to matingly engage an external surface of the pull rod such that rotation of the plunger relative to the implant main body causes rotation and release of the pull rod from the implant main body.

Statement 17: A wound closure device according to Statement 16, wherein the aperture surface of the plunger and the external surface of the pull rod are each hexagonal.

Statement 18: A wound closure device according to Statement 16 or Statement 17, wherein the plunger comprises a ring collar.

Statement 19: A wound closure device according to any one of the preceding Statements 1-18; wherein the applicator comprises an engagement mechanism, wherein the plurality of tissue engagement hooks are configured to penetrate at least one layer of body tissue upon actuation of the engagement mechanism.

Statement 20: A wound closure device according to Statement 19, wherein the engagement mechanism comprises a plurality of tethers each coupled with a corresponding one of the plurality of tissue engagement hooks.

Statement 21: A wound closure device according to Statement 20, wherein each of the plurality of tethers is looped.

Statement 22: A wound closure device according to Statement 20 or Statement 21, wherein the plurality of tethers are configured to cause the plurality of tissue engagement hooks to penetrate at least one layer of body tissue upon the application of tension to the plurality of tethers.

Statement 23: A wound closure device according to any one of the preceding Statements 20-22, wherein the engagement mechanism comprises a plunger and a detent, wherein the plunger is configured to apply tension to the plurality of tethers upon release of the detent from engagement with the applicator main body.

Statement 24: A wound closure device according to Statement 23, wherein the plunger comprises a ring collar.

Statement 25: A wound closure device according to any one of the preceding Statements 20-24, wherein the applicator further comprises one or more apertures configured to provide sufficient access to the plurality of tethers so that the plurality of tethers can be severed.

Statement 26: A wound closure device according to Statement 25, wherein the plurality of tethers are formed in one or more loops.

Statement 27: A wound closure device according to Statement 26, wherein a single applicator aperture provides sufficient access to the plurality of tethers so that the plurality of tethers can be severed.

Statement 28: A wound closure device according to any of the preceding Statements 20-28, wherein the applicator comprises a pull rod and wherein the pull rod is configured to cause the release of the applicator from the implant main body upon rotation of the pull rod relative to the implant main body.

Statement 29: A wound closure device according to Statement 28, wherein the plurality of tethers are configured to be severed upon rotation of the pull rod relative to the implant main body.

Statement 30: A wound closure device according to Statement 24, wherein the plurality of tethers are configured to be severed upon rotation of the ring collar relative to the implant main body.

Statement 31: A wound closure device according to any one of the preceding Statements 19-30, wherein the engagement mechanism is configured to cause the release of the applicator from the implant main body upon mechanical release of the engagement mechanism, or a portion thereof, relative to the implant main body.

Statement 32: A wound closure device according to Statement 31, wherein the engagement mechanism is configured to mechanically release upon rotation of the engagement mechanism, or a portion thereof, relative to the implant main body.

Statement 33: A wound closure device according to any one of the preceding Statements 19-32, wherein the engagement mechanism comprises a plunger and a pull rod, wherein the plunger includes an aperture having a surface configured to matingly engage an external surface of the pull rod such that rotation of the plunger relative to the implant main body causes rotation and release of the pull rod from the implant main body.

Statement 34: A wound closure device according to Statement 33, wherein the aperture surface of the plunger and the external surface of the pull rod are each hexagonal.

Statement 35: A wound closure device according to Statement 33 or Statement 34, wherein the plunger comprises a ring collar.

Statement 36: A wound closure device according to any one of the preceding Statements 1-35, wherein the applicator comprises an extension assembly, wherein the extension assembly, upon actuation, is configured to extend the plurality of connecting members from the implant main body.

Statement 37: A wound closure device according to Statement 36, wherein the extension assembly comprises a linkage, wherein the linkage, upon actuation, is configured to extend the plurality of connecting members from the implant main body.

Statement 38: A wound closure device according to Statement 37, wherein the linkage is a push wire.

Statement 39: A wound closure device according to any one of the preceding Statements 1-38, wherein the plurality of connecting members each comprises a spring deployment retainer configured to capture a proximal end of a corresponding one of the plurality of tissue engagement hooks after being moved to an engaged configuration.

Statement 40: A wound closure device according to Statement 39, wherein the spring deployment retainer is in the form of a receiving space formed in the corresponding one of the plurality of connecting members, and the proximal end of the one of the plurality of tissue engagement hooks is configured to expand upon being placed within the receiving space, thereby preventing the hook to return to a state prior to the engaged configuration.

Statement 41: A wound closure device according to Statement 40, wherein the proximal end of the one of the plurality of tissue engagement hooks enters the receiving space upon application of tension to the plurality of tethers whereby the proximal end of the one of the plurality of tissue engagement hooks is captured by a portion of the receiving space that prevents translational movement of the corresponding tissue engagement hook.

Statement 42: A wound closure device according to any one of the preceding Statements 1-38, wherein the plurality of tissue engagement hooks each comprise one or more rotating hooks coupled with a biasing member, the one or more rotating hooks held in an unactuated condition by a retention device coupled with a tether, and wherein the rotating hook is configured to penetrate at least one layer of body tissue upon application of tension to the tether thereby disengaging the retention device and allowing the biasing member to cause the rotation of the corresponding hook to engage the tissue.

Statement 43: A wound closure device according to Statement 42, wherein the biasing member is a torsion spring.

Statement 44: A wound closure device according to Statement 42 or Statement 43, wherein the retention device is a lock-out pin.

Statement 45: A wound closure device according to any one of the preceding Statements 1-38, wherein the plurality of tissue engagement hooks each comprise a pre-curled hook, which in an deployed and unbiased state is curled and in a retracted and biased state is deformed such that the curled portion has less of a curl or no curl.

Statement 46: A wound closure device according to Statement 45, wherein each pre-curled hook is elastically deformed within an interior portion of the connecting member, slidably disposed about the connecting member and coupled with a corresponding one of the plurality of tethers, wherein each pre-curled hook is configured to penetrate at least one layer of body tissue upon application of tension to the tether, causing the pre-curled hook to slide outward from within the interior portion of the connecting member.

Statement 47: A method for closing a fascial wound on a patient comprising: inserting a wound closure device into an abdominal cavity partially beyond a location of the fascial wound, wherein the wound closure device comprises: an applicator releasably engaged with an implant main body; a plurality of connecting members, each configured to extend from the implant main body; and a plurality of tissue engagement hooks coupled with a respective one of the plurality of connecting members; extending the plurality of connecting members from the implant main body such that the connecting members rotate about the implant main body; bringing the plurality of tissue engagement hooks into pressure contact with at least one layer of tissue; causing the plurality of tissue engagement hooks to penetrate at least one layer of body tissue; and disengaging the applicator from the implant main body.

Statement 48: A method for closing a fascial wound on a patient according to Statement 47, wherein the wound closure device is configured such that during insertion into the abdominal cavity the plurality of tissue engagement hooks are closer to a proximal end of the applicator than when engaged with the at least one layer of tissue.

Statement 49: A method for closing a fascial wound on a patient according to Statement 47 or Statement 48, wherein extending the plurality of connecting members from the implant main body comprises actuating the applicator.

Statement 50: A method for closing a fascial wound on a patient according to Statement 47 or Statement 48, wherein extending the plurality of connecting members from the implant main body comprises retracting a securement member partially surrounding the plurality of connecting members.

Statement 51: A method for closing a fascial wound on a patient according to Statement 47 or Statement 48, wherein extending the plurality of connecting members from the implant main body comprises retracting a retractable sleeve.

Statement 52: A method for closing a fascial wound on a patient according to Statement 47 or Statement 48, wherein extending the plurality of connecting members from the implant body comprises translating a retractable sleeve to engage the plurality of connecting members.

Statement 53: A method for closing a fascial wound on a patient according to Statement 47 or Statement 48, wherein the extending the plurality of connecting members from the implant body comprises applying a deployment motion to a linkage of the applicator.

Statement 54: A method for closing a fascial wound on a patient according to any one of the preceding Statements 47-53, wherein bringing the plurality of tissue engagement hooks into pressure contact with at least one layer of tissue comprises applying a pulling motion to the applicator.

Statement 55: A method for closing a fascial wound on a patient according to any one of the preceding Statements 47-53, wherein bringing the plurality of tissue engagement hooks into pressure contact with at least one layer of tissue comprises activating an engagement mechanism on the applicator.

Statement 56: A method for closing a fascial wound on a patient according to any one of the preceding Statements 47-53, wherein bringing the plurality of tissue engagement hooks into pressure contact with at least one layer of tissue comprises retracting the applicator.

Statement 57: A method for closing a fascial wound on a patient according to any one of the preceding Statements 47-53, wherein bringing the plurality of tissue engagement hooks into pressure contact with at least one layer of tissue comprises applying a pulling motion on a ring collar on the applicator.

Statement 58: A method for closing a fascial wound on a patient according to any one of the preceding Statements 47-53, wherein bringing the plurality of tissue engagement hooks into pressure contact with at least one layer of tissue comprises applying tension to a plurality of tethers coupled to a corresponding one of the plurality of tissue engagement hooks.

Statement 59: A method for closing a fascial wound on a patient according to any one of the preceding Statements 47-58, wherein causing the plurality of tissue engagement hooks to penetrate at least one layer of body tissue comprises applying a pulling motion to the applicator.

Statement 60: A method for closing a fascial wound on a patient according to any one of the preceding Statements 47-58, wherein causing the plurality of tissue engagement hooks to penetrate at least one layer of body tissue comprises activating an engagement mechanism on the applicator.

Statement 61: A method for closing a fascial wound on a patient according to any one of the preceding Statements 47-58, wherein causing the plurality of tissue engagement hooks to penetrate at least one layer of body tissue comprises retracting the applicator.

Statement 62: A method for closing a fascial wound on a patient according to any one of the preceding Statements 47-58, wherein causing the plurality of tissue engagement hooks to penetrate at least one layer of body tissue comprises applying tension to a plurality of tethers coupled to a corresponding one of the plurality of tissue engagement hooks.

Statement 63: A method for closing a fascial wound on a patient according to Statement 62, wherein applying tension to a plurality of tethers comprises pulling a ring collar on the applicator until a detent releases the ring collar.

Statement 64: A method for closing a fascial wound on a patient according to any one of the preceding Statements 47-63, wherein disengaging the applicator from the implant main body comprises rotating the applicator relative to the implant main body.

Statement 65: A method for closing a fascial wound on a patient according to any one of the preceding Statements 47-63, wherein disengaging the applicator from the implant main body comprises rotating a ring collar on the applicator relative to the implant main body.

Statement 66: A method for closing a fascial wound on a patient according to any one of the preceding Statements 47-63, wherein disengaging the applicator from the implant main body comprises rotating a pull rod on the applicator relative to the implant main body.

Statement 67: A method for closing a fascial wound on a patient according to any one of the preceding Statements 47-66, further comprising severing a plurality of tethers coupled with the plurality of tissue engagement hooks.

Statement 68: A method for closing a fascial wound on a patient according to Statement 67, wherein severing a plurality of tether occurs upon disengagement of the applicator from the implant body.

Statement 69: A method for closing a fascial wound on a patient according to Statement 67, wherein severing a plurality of tethers occurs upon rotation of a pull rod on the applicator relative to the implant main body.

Statement 70: A method for closing a fascial wound on a patient according to Statement 67, wherein severing a plurality of tethers occurs upon rotation of a ring collar on the applicator relative to the implant main body.

Statement 71: A method for closing a fascial wound on a patient according to any one of the preceding Statements 47-70, wherein disengaging the applicator from the implant main body comprises severing a plurality of tethers coupled with the plurality of tissue engagement hooks.

Statement 72: A method for closing a fascial wound on a patient according to any one of the preceding Statements 47-71, wherein disengaging the applicator from the implant main body comprises activating a disengagement mechanism on the applicator that simultaneously or sequentially severs one or more tethers and mechanically releases the applicator from the implant main body.

Statement 73: A method for closing a fascial wound on a patient according to Statement 72, wherein activating a disengagement mechanism on the applicator comprises rotating a pull rod on the applicator relative to the implant main body.

Statement 74: A method for closing a fascial wound on a patient according to Statement 72, wherein activating a disengagement mechanism on the applicator comprises pulling a plunger on the applicator.

Statement 75: A method for closing a fascial wound on a patient according to any one of the preceding Statements 47-74, wherein disengaging the applicator from the implant main body comprises activating a disengagement mechanism on the applicator that simultaneously or sequentially severs one or more tethers and rotates a pull rod on the applicator to mechanically release the applicator from the implant main body.

Statement 76: A wound closure device comprising: an applicator having a main body and a distal end and a proximal end, wherein the distal end is configured to releasably engage an implant main body; a securement member coupled to the applicator and having a retractable sleeve; a plurality of elastic members coupled to the implant main body, each of the plurality of elastic members having a tissue engagement hook formed at a distal end thereof, wherein in a first configuration the plurality of tissue engagement hooks are retained by the retractable sleeve and are closer to the implant main body than a second configuration in which the plurality of elastic members are biased outward from the implant main body, so that the plurality of tissue engagement hooks are located away from the implant main body.

Statement 77: A wound closure device according to Statement 76, further comprising one or more tethers coupled to corresponding ones of the plurality of tissue engagement hooks, wherein the one or more tethers are configured to adjust deployment of the plurality of tissue engagement hooks, so that from the second configuration the hooks can be returned to be closer to the applicator.

Statement 78: A wound closure device according to Statement 76 or Statement 77, wherein the plurality of tissue engagement hooks have a U-shaped portion such that the distal end of the plurality of tissue engagement hooks is configured to contact the tissue first.

Statement 79: A wound closure device according to Statement 78, wherein the U-shaped portion is configured to receive a corresponding one of the one or more tethers.

Statement 80: A wound closure device according to Statement 79, wherein the one or more tethers is a loop of material.

Statement 81: A wound closure device according to any one of the preceding Statements 76-80, wherein the plurality of elastic members are at least partially wound around the applicator main body in the first configuration.

Statement 82: A wound closure device according to any one of the preceding Statements 76-81, wherein the plurality of elastic members number four and the plurality of tissue engagement hooks number four.

Statement 83: A wound closure device according to any one of the preceding Statements 76-82, wherein the applicator main body has a plurality of apertures formed therethrough, such that each of the plurality of apertures correspond to one of the plurality of tissue engagement hooks.

Statement 84: A wound closure device according to Statement 83, wherein the plurality of apertures are substantially an upside down U-shaped aperture.

Statement 85: A wound closure device according to any one of the preceding Statements 76-84, wherein at least one of the plurality of elastic members is partially wound around the implant main body in the first configuration.

Statement 86: A wound closure device according to any one of the preceding Statements 76-85, further comprising an engagement mechanism that is coupled to the implant main body.

Statement 87: A wound closure device according to Statement 86, wherein the engagement mechanism comprises a pull rod.

Statement 88: A wound closure device according to Statement 87, wherein the engagement mechanism has a distal end that is threaded and configured to threadingly engage with the implant main body, so that the pull rod can be disengaged from the implant main body once the implant main body is installed in the patient.

Although a variety of examples and other information was used to explain aspects within the scope of the appended claims, no limitation of the claims should be implied based on particular features or arrangements in such examples, as one of ordinary skill would be able to use these examples to derive a wide variety of implementations. Further and although some subject matter may have been described in language specific to examples of structural features and/or method steps, it is to be understood that the subject matter defined in the appended claims is not necessarily limited to these described features or acts. For example, such functionality can be distributed differently or performed in components other than those identified herein. Rather, the described features and steps are disclosed as examples of components of systems and methods within the scope of the appended claims. Moreover, claim language reciting “at least one of” a set indicates that a system including either one member of the set, or multiple members of the set, or all members of the set, satisfies the claim.

1. A wound closure device comprising:
an applicator having a distal end and a proximal end, wherein the distal end is configured to releasably engage an implant main body;
a plurality of connecting members, each configured to extend from the implant main body; and
a plurality of tissue engagement hooks, each of the plurality of tissue engagement hooks being mounted within a respective one of the plurality of connecting members;
wherein the plurality of connecting members rotate about the implant main body, so that in a retracted configuration the plurality of tissue engagement hooks are closer to the proximal end of the applicator than in an engaged configuration.

2.-5. (canceled)

6. The wound closure device according to claim 1, wherein the applicator comprises an engagement mechanism, wherein the plurality of tissue engagement hooks are configured to penetrate at least one layer of body tissue upon actuation of the engagement mechanism.

7. The wound closure device according to claim 6, wherein the engagement mechanism comprises a plurality of tethers each coupled with a corresponding one of the plurality of tissue engagement hooks.

8. The wound closure device according to claim 7, wherein each of the plurality of tethers is looped.

9. The wound closure device according to claim 8, wherein the plurality of tethers are configured to cause the plurality of tissue engagement hooks to penetrate at least one layer of body tissue upon the application of tension to the plurality of tethers.

10. The wound closure device according to claim 9, wherein the engagement mechanism comprises a plunger and a detent, wherein the plunger is configured to apply tension to the plurality of tethers upon release of the detent from engagement with the applicator main body.

11. The wound closure device according to claim 10, wherein the plunger comprises a ring collar.

12. The wound closure device according to claim 10, wherein the applicator further comprises one or more apertures configured to provide sufficient access to the plurality of tethers so that the plurality of tethers can be severed.

13. The wound closure device according to claim 12, wherein the plurality of tethers are formed in one or more loops.

14. The wound closure device according to claim 13, wherein a single applicator aperture provides sufficient access to the plurality of tethers so that the plurality of tethers can be severed.

15. The wound closure device according to claim 6, wherein the applicator comprises a pull rod and wherein the pull rod is configured to cause the release of the applicator from the implant main body upon rotation of the pull rod relative to the implant main body.

16. The wound closure device according to claim 15, wherein the plurality of tethers are configured to be severed upon rotation of the pull rod relative to the implant main body.

17. The wound closure device according to claim 11, wherein the plurality of tethers are configured to be severed upon rotation of the ring collar relative to the implant main body.

18. The wound closure device according to claim 6, wherein the engagement mechanism comprises a plunger and a pull rod, wherein the plunger includes an aperture having a surface configured to matingly engage an external surface of the pull rod such that rotation of the plunger relative to the implant main body causes rotation and release of the pull rod from the implant main body.

19. The wound closure device according to claim 18, wherein the aperture surface of the plunger and the external surface of the pull rod are each hexagonal.

20. The wound closure device according to claim 18, wherein the plunger comprises a ring collar.

21.-25. (canceled)

26. The wound closure device according to claim 1, wherein the plurality of connecting members each comprises a spring deployment retainer configured to capture a proximal end of a corresponding one of the plurality of tissue engagement hooks after being moved to an engaged configuration.

27.-33. (canceled)

34. A method for closing a fascial wound on a patient comprising:
inserting a wound closure device into an abdominal cavity partially beyond a location of the fascial wound, wherein the wound closure device comprises:
an applicator releasably engaged with an implant main body;
a plurality of connecting members, each configured to extend from the implant main body; and
a plurality of tissue engagement hooks coupled with a respective one of the plurality of connecting members;
extending the plurality of connecting members from the implant main body such that the connecting members rotate about the implant main body;
bringing the plurality of tissue engagement hooks into pressure contact with at least one layer of tissue;
causing the plurality of tissue engagement hooks to penetrate at least one layer of body tissue; and
disengaging the applicator from the implant main body.

35.-39. (canceled)

40. The method according to claim 34, wherein the extending the plurality of connecting members from the implant main body comprises applying a deployment motion to a linkage of the applicator.

41.-62. (canceled)

63. A wound closure device comprising:
an applicator having a main body and a distal end and a proximal end, wherein the distal end is configured to releasably engage an implant main body;
a securement member coupled to the applicator and having a retractable sleeve;
a plurality of elastic members coupled to the implant main body, each of the plurality of elastic members having a tissue engagement hook formed at a distal end thereof, wherein in a first configuration the plurality of tissue engagement hooks are retained by the retractable sleeve and are closer to the implant main body than a second configuration in which the plurality of elastic members are biased outward from the implant main body, so that the plurality of tissue engagement hooks are located away from the implant main body.

64.-75. (canceled)
